(19) United States
(12) Patent Application Publication
(10) Pub. No.: US 2020/0017811 A1
(43) Pub. Date: Jan. 16, 2020

(54) System for Obtaining 3D Micro-Tissues

(71) Applicant: SageMedic Corporation, Redwood City, CA (US)

(72) Inventor: Christian Apfel, Larkspur, CA (US)

(21) Appl. No.: 16/583,145

(22) Filed: Sep. 25, 2019

Related U.S. Application Data
(63) Continuation of application No. 16/102,652, filed on Aug. 13, 2018.
(60) Provisional application No. 62/686,563, filed on Jun. 18, 2018.

(51) Int. Cl. C12M 3/00; C12M 1/32

(52) U.S. Cl. CPC C12M 21/08; G01N 33/5008; C12M 23/12

(57) ABSTRACT

Provided are systems and methods for obtaining a three-dimensional micro-tissue. For example, the system may include at least one cell culture device that may include one or more wells. Each well may include an opening at an upper surface of the cell culture device. Each well may include a bottom located towards a lower surface of the cell culture device. The bottom may be characterized by a bottom surface area inside each well. Each well may include a wall extending from the opening to the bottom surface area to define a total volume. Each well may be characterized by an aggregation factor greater than 800.

### FIELD OF INVENTION

The present application relates to devices containing at least one well for cell culture, methods for creating three-dimensional micro-tissues from cells, and such three-dimensional micro-tissues.

### BACKGROUND

Multi-well plates are used in life science cell assays to culture cells to study, for example, metabolic pathways, gene and protein expressions, toxicological effects, and drug discovery.

Methods for assaying live cancer cells are desirable for both drug development and treatment selection for cancer patients. The most effective treatment for an individual patient is generally unknown. For example, the choice of drug is a hit or miss experiment with tumor response rates for metastatic lung cancer patients being as low as 30% or less.

One approach is to seed a suspension of a patient's tumor cells in multi-well plates. The cells sediment to the bottom of the plate and grow in a 2-dimensional mono-layer, which may be exposed to one or more drugs to determine inhibition of cell growth or induction of cell death (cell behavior). Such 2D approaches may be of limited use because physiological cell behavior that depends upon 3D effects is omitted, such as the effects of cell-cell interactions, and concentration gradients of gases, metabolites, and chemical entities.

In principle, live, single cancer cells have the ability to self-aggregate, establish new cell-cell interactions, and form 3-dimensional cancer tissues. Round bottom well plates with special coatings have been used to form three-dimensional spheroids by sedimentation of cells. Tumor cell spheroids may also be formed with a hanging drop method.

Unfortunately, present 3D approaches are also of limited use. The hanging drop method, for example, may be labor intensive, requiring manual pipetting, and may be easily disrupted by vibration. The hanging drop method also uses additional fluid filled regions to address evaporation and spheroid collection issues. Further, most of the cells may be exposed to unnaturally high concentrations of oxygen, nutrients, and drugs, except for the cells in the core of the spheroid, which, being in the core, may be difficult to image at a desired resolution. Moreover, round bottom well plates may be particularly problematic for imaging because of light dispersion. Also, to date, 3D methods have only been shown to work reliably with highly processed, immortal cancer cell lines such as HeLa, in contrast to primary tumor cell lines from individual patients.

The present application appreciates that conducting cell-based assays may be a challenging endeavor.

### SUMMARY OF THE INVENTION

In various embodiments, a system for obtaining a three-dimensional micro-tissue is provided. The system may include a cell culture device. The cell culture device may include a plurality of wells. Each well may include an opening at an upper surface of the device. The opening may be characterized by an opening cross-sectional area. Each well may include a bottom located towards a lower surface of the device. The bottom may be characterized by a bottom surface area inside the well. The bottom surface area may include a planar portion. The bottom may be characterized by transparency effective to permit imaging or spectroscopy inside each well, e.g., from below the lower surface of the cell culture device. Each well may include a wall extending from the opening to the bottom. The wall may define a total volume between the opening and the bottom. The wall may define a neck located below the opening. The well may define a neck characterized by a neck cross-sectional area parallel to the opening. The wall may define a concentrating volume between the neck and the opening. The wall may define a culturing volume between the neck and the bottom. Each well may be characterized by an aggregation factor of greater than 800. The aggregation factor may correspond to the total volume divided by the bottom surface area divided by a unit length.

In various embodiments, a method for characterizing a three-dimensional micro-tissue is provided. The method may include providing a system for obtaining a three-dimensional micro-tissue. The system may include a cell culture device. The cell culture device may include a plurality of wells. Each well may include an opening at an upper surface of the device. The opening may be characterized by an opening cross-sectional area. Each well may include a bottom located towards a lower surface of the device. The bottom may be characterized by a bottom surface area inside the well. The bottom surface area may include a planar portion. The bottom may be characterized by transparency effective to permit imaging or spectroscopy inside each well, e.g., from below the lower surface of the cell culture device. Each well may include a wall extending from the opening to the bottom. The wall may define a total volume between the opening and the bottom. The wall may define a neck located below the opening. The well may define a neck characterized by a neck cross-sectional area parallel to the opening. The wall may define a concentrating volume between the neck and the opening. The wall may define a culturing volume between the neck and the bottom. Each well may be characterized by an aggregation factor of greater than 800. The aggregation factor may correspond to the total volume divided by the bottom surface area divided by a unit length. The method may include providing each well with a suspension of cells, cell clusters, and/or tissue fragments. The method may include aggregating the cells, cell clusters, and/or tissue fragments from the suspension to a bottom surface area according to the aggregation factor. The aggregating may be effective to obtain the three-dimensional micro-tissue. The method may include characterizing the three-dimensional micro-tissue inside each well from below the lower surface of the cell culture device.

In various embodiments, a method for obtaining a three-dimensional micro-tissue is provided. The method may include providing a system for obtaining a three-dimensional micro-tissue. The system may include a cell culture device. The cell culture device may include a plurality of wells. Each well may include an opening at an upper surface of the device. The opening may be characterized by an opening cross-sectional area. Each well may include a bottom located towards a lower surface of the device. The bottom may be characterized by a bottom surface area inside the well. The bottom surface area may include a planar portion. The bottom may be characterized by transparency effective to permit imaging or spectroscopy inside each well, e.g., from below the lower surface of the cell culture device. Each well may include a wall extending from the opening to the bottom. The wall may define a total volume between the opening and the bottom. The wall may define a neck located below the opening. The well may define a neck characterized by a neck cross-sectional area parallel to the opening. The wall may define a concentrating volume between the neck and the opening. The wall may define a culturing volume between the neck and the bottom. Each well may be characterized by an aggregation factor of greater than 800. The aggregation factor may correspond to the total volume divided by the bottom surface area divided by a unit length. The method may include providing each well with a suspension of cells, cell clusters, and/or tissue fragments. The method may include aggregating the cells, cell clusters, and/or tissue fragments from the suspension to a bottom surface area according to the aggregation factor. The aggregating may be effective to obtain the three-dimensional micro-tissue.

Various features, aspects, and advantages of the present invention will become more apparent from the following detailed description of preferred embodiments of the invention, along with the accompanying drawings in which like numerals represent like components.

### BRIEF DESCRIPTION OF THE FIGURES

FIG. 1A is a side cross-section view of an example system 100.

FIG. 1B is a top view of example system 100.

FIG. 1C is a sketch in vertical cross-section showing that wall 118 may define a truncated paraboloid that decreases in diameter from opening 106 towards neck 126.

FIG. 1D is a sketch in vertical cross-section showing that wall 118 may define a truncated hyperboloid that decreases in diameter from opening 106 towards neck 126.

FIG. 1E is a sketch in vertical cross-section showing that wall 118 may define a hyperboloid that extends from opening 106 to bottom 110, with neck 126 located at the vertex of the hyperboloid.

FIG. 1F is a sketch in vertical cross-section showing that wall 118 may define two truncated sections, e.g., truncated cones, that each decrease in diameter from opening 106 towards neck 126.

FIG. 1G is a sketch in vertical cross-section showing that wall 118 may define a truncated cone that decreases in diameter from bottom 110 towards neck 126.

FIG. 1H is a sketch in vertical cross-section showing that wall 118 may define a truncated paraboloid that decreases in diameter from bottom 110 towards neck 126.

FIG. 1I is a sketch in vertical cross-section showing that wall 118 may define a truncated hyperboloid that decreases in diameter from bottom 110 towards neck 126.

FIG. 1J is a perspective drawing showing cell culture device 102 as a multi-well plate with a regular array of 8×12=96 wells, held in frame 124.

FIG. 2 is a perspective view of an example system 200.

### DETAILED DESCRIPTION

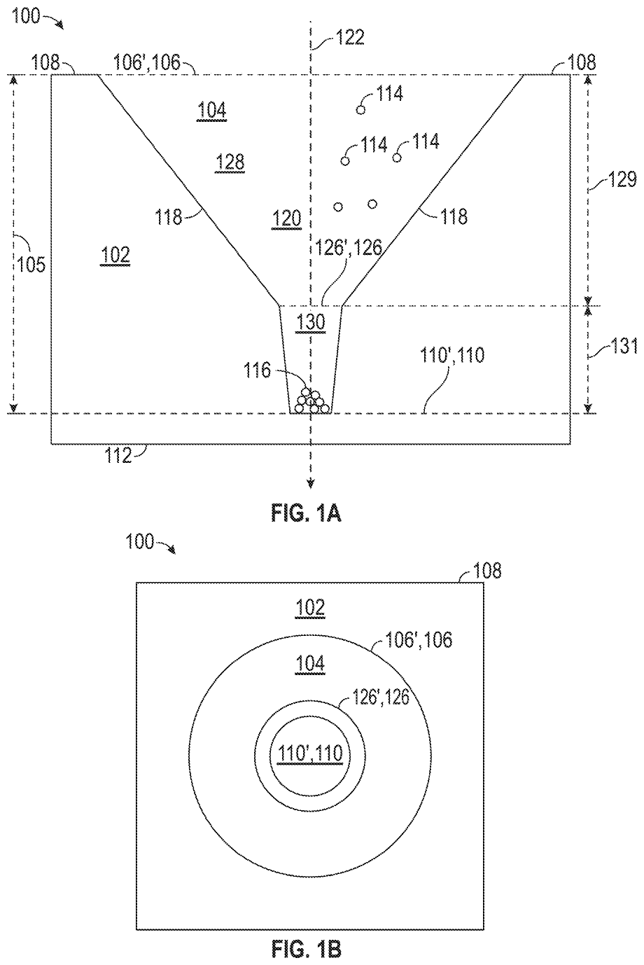

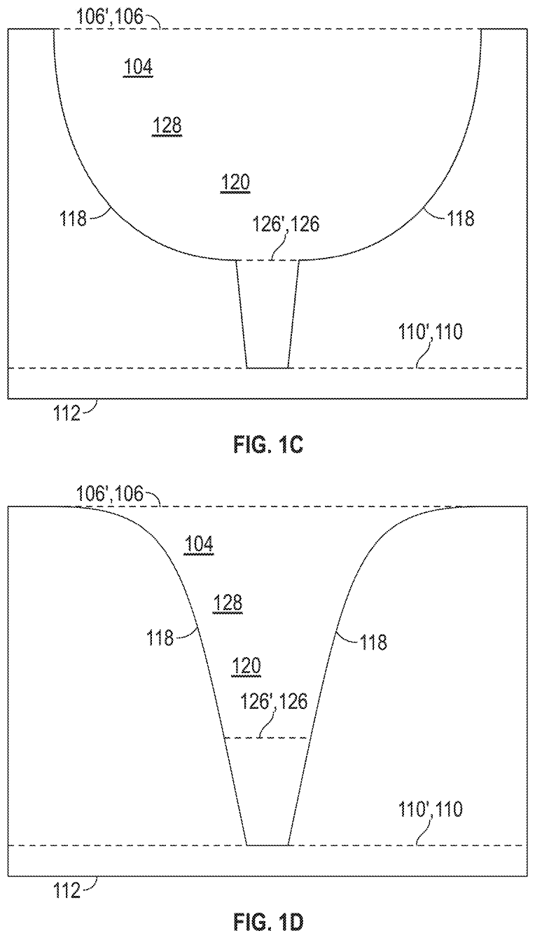

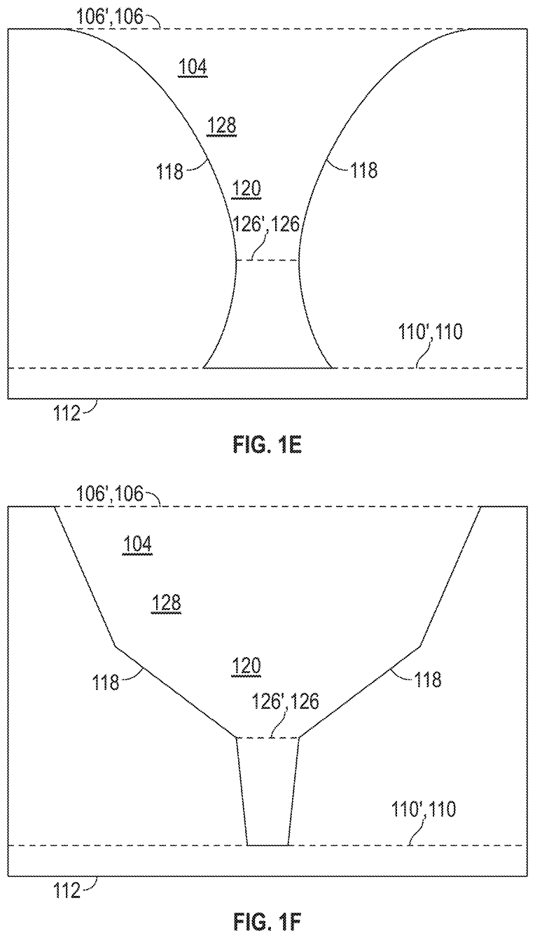

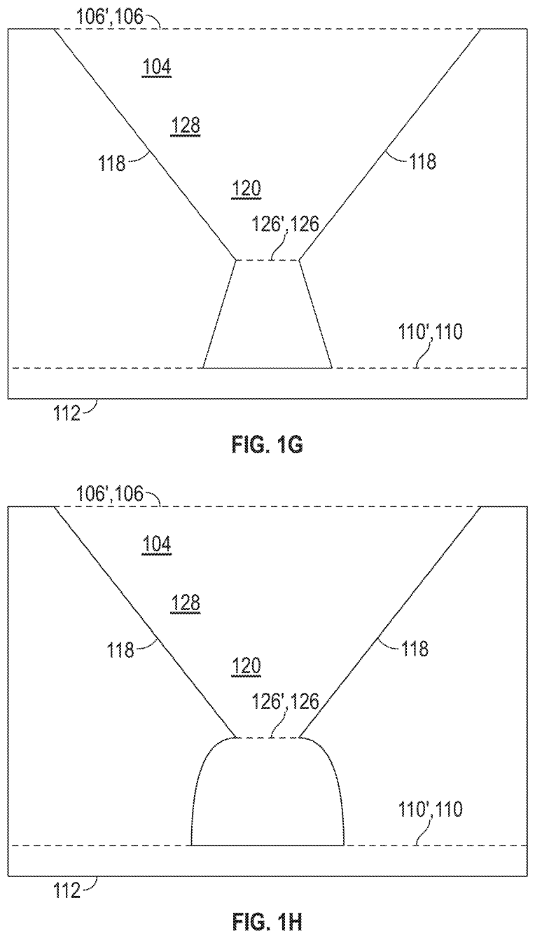

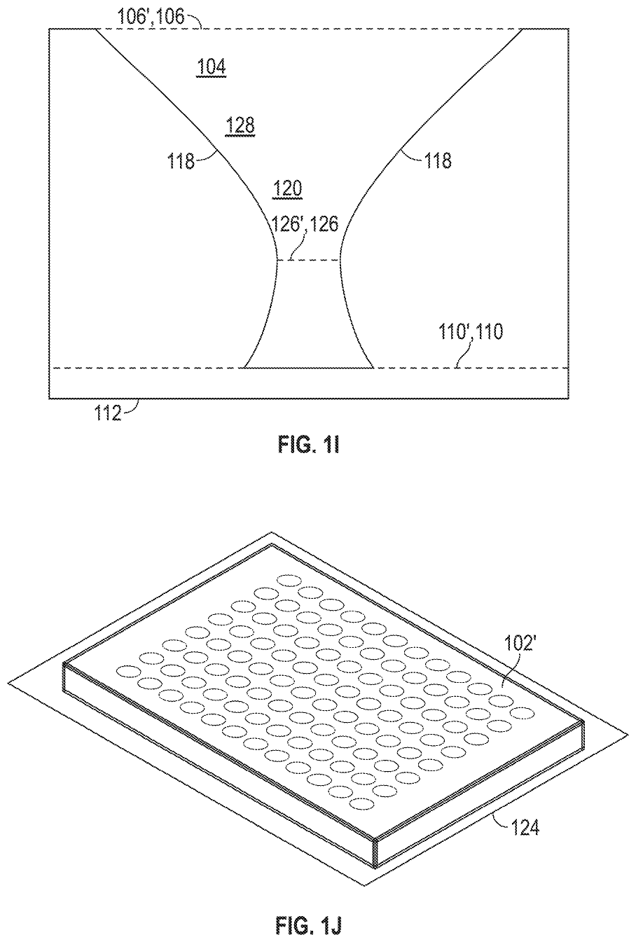

In various embodiments, a system for obtaining a three-dimensional micro-tissue, e.g., for characterization, is provided. FIG. 1A is a side cross-section view of an example system 100. FIG. 1B is a top view of example system 100. System 100 may include at least one cell culture device 102. Cell culture device 102 may include one or more wells 104. For example, cell culture device 102 may be a well plate or a microfluidic plate. Each well 104 may include an opening 106 at an upper surface 108 of cell culture device 102. Each well 104 may include a bottom 110 located towards a lower surface 112 of cell culture device 102. Bottom 110 may be characterized by a bottom surface area 110′ inside well 104. Bottom surface area 110′ may be configured to receive aggregating cells, cell clusters, and/or tissue fragments 114 to form three-dimensional micro-tissue 116. Each well 104 may include a wall 118 extending from opening 106 to bottom surface area 110′. Wall 118 may define a total volume 120 between opening 106 and bottom surface area 110′. Each well 104 may be characterized by a well height 105.

Each well 104 may be characterized by an aggregation factor greater than 100. The aggregation factor and a corresponding notional aggregation height may be related to total volume divided by a surface area that receives aggregating cells, cell clusters, and/or tissue fragments, e.g., total volume 120 divided by bottom surface area 110′. Because volume divided by area results in units of length, total volume 120 divided by bottom surface area 110′ may be contemplated in units of length characteristic of each well 104, e.g., the notional aggregation height. The aggregation height may be converted to the aggregation factor, which is unitless, by dividing by unit length. For example, an aggregation height of 100 mm divided by unit length of 1 mm corresponds to a unitless aggregation factor of 100. Each well may be characterized by the aggregation factor at a value of about, at least about, or greater than one of: 100, 125, 150, 175, 200, 250, 300, 400, 500, 600, 700, 750, 800, 900, 1,000, 1,500, 2,000, 2,500, 5,000, 7,500, 10,000, 15,000, 20,000, 30,000, 40,000, 50,000, 75,000, or 100,000, or a range between any two of the preceding values, for example, between greater than 100 and about 100,000, between about 200 and about 100,000, between about 500 and about 50,000, between about 800 and about 40,000, between about 800 and about 75,000, and the like.

Each well 104 may be described with reference to vertical well axis 122. Vertical well axis 122 may extend from a centroid of opening 106 in a direction down through a centroid of bottom 110. Wall 118 may extend in rotational symmetry about vertical well axis 122. One or more of upper surface 108, lower surface 112, bottom 110, and bottom surface area 110′ may be horizontal with respect to vertical well axis 122.

Wall 118 may define a neck 126 located below opening 106. Neck 126 may be characterized by a neck cross-sectional area 126′ that is horizontal, e.g., parallel to opening 106 and perpendicular to vertical well axis 122. Wall 118 may define a concentrating volume 128 between neck 126 and opening 106. Concentrating volume 128 may be characterized by a value in μL of one of about, or at least one of about: 10, 15, 20, 25, 30, 40, 50, 60, 70, 80, 90, 100, 125, 150, 175, 200, 225, 250, 500, 750, 1,000, 1,250, and 1,500, or a range between any two of the preceding values, for example, between about: 10-1,500 μL, 10-1,000 μL, 10-250 μl, 50-225 μL, 10-200 μL, 25-125 μL, 50-100 μL, and the like.

Each well 104 may be defined in concentrating volume 128 by a concentrating volume height 129, e.g., between opening 106 and neck 126 along vertical well axis 122. Concentrating volume height 129 in concentrating volume 128 may have a value in mm of one of about, or at least one of about: 0.5, 1, 1.5, 2, 2.5, 3, 4, 5, 6, 7, 7.5, 8, 9, 10, 11, 12, 12.5, 13, 14, 15, 17.5, 20, 22.5, and 25, or a range between any two of the preceding values, for example, between about: 0.5-25 mm, 0.5-20 mm, 1-15 mm, 2-12 mm, 2-10 mm, 2-4 mm, and the like. For example, each well 104 may be defined by a concentrating volume height 129 between opening 106 and neck 126 along vertical well axis 122 of at least about 0.5 mm.

As used herein, a “neck” defined by a wall in a well is in a horizontal plane with respect to a vertical well axis, the horizontal plane being located below a well opening. For example, neck 126 may be defined by wall 118 in well 104 in a horizontal plane with respect to vertical well axis 122 below opening 106, the horizontal plane of neck 126 being coincident with neck cross-sectional area 126′.

In some embodiments, each neck 126 may be located at a narrowest portion of each well 104 as defined by wall 118 below opening 106. In some embodiments, the narrowest portion of each well 104 may be a portion of constant diameter that extends along well axis 122 for a distance, and neck 126 may be located at the uppermost extent of the portion of constant diameter, closest to opening 106.

In some embodiments, each neck 126 may be located at a first significant narrowing of a horizontal dimension of well 104 below opening 106, as depicted in FIG. 1A. For example, wall 118 in a vertical cross-section of well 104 may describe a two-dimensional function with vertical well axis 122 corresponding to a y axis extending vertically from a zero origin at an intersection of vertical well axis 122 and well bottom 110, and an x axis of the function extending horizontally from a zero origin at the intersection of vertical well axis 122 and well bottom 110. For the two-dimensional function described over x>0 by wall 118, neck 126 may be located where one of: a first derivative is 1; the first derivative is infinite; the first derivative is at a local maximum; the first derivative is at a global maximum over x>0; the first derivative is undefined and the function is discontinuous; a second derivative is undefined and the function is discontinuous; the second derivative is at a local minimum; the second derivative is at a global minimum over x>0; the second derivative is at a local maximum; and the second derivative is at a global maximum over x>0. In some embodiments, wall 118 may extend down from opening 106 in the form of a cylinder, or in a decreasing diameter to define a cone, a paraboloid, or a hyperboloid. Neck 126 may be located at a lower truncation of the cone, paraboloid, or hyperboloid. Wall 118 may define two or more such shapes, for example, wall 118 may define a cylinder extending down from opening 106, and a cone truncated at neck 126 or bottom 110, the cone increasing in diameter and extending upwards to meet the cylinder.

In some embodiments, wall 118 may extend between neck 126 and bottom 110 in each well 104. Wall 118 in each well 104 may define a culturing volume 130 between neck 126 and bottom 110. For example, neck 126 and bottom 110 may be separated by a culturing volume height 131 along vertical well axis 122. Culturing volume height 131 may have a value in mm of one of, or one of about: 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2, or a range between any two of the preceding values, for example, from about 0.001 mm to about 2 mm.

Culturing volume 130 may be characterized by a value in μL of one of about, or at least one of about: 1, 2, 2.5, 3, 4, 5, 6, 7, 7.5, 8, 9, 10, 15, 20, 25, 30, 40, 50, 60, 70, 80, 90, 100, 125, 150, 175, 200, 225, and 250, or a range between any two of the preceding values, for example, between about: 1-250 μL, 1-200 μL, 1-150 μL, 1-50 μL, 1-25 μl, and the like. For example, culturing volume 130 may be between about 1 μL and about 250 μL.

Each well 104 may be characterized by a ratio of concentrating volume 128 to culturing volume 130 that is one of at least about 10:1, 15:1, 20:1, 25:1, 50:1, 75:1, 100:1, 250:1, 500:1, 750:1, 1,000:1, 1,500:1, 2,500:1, 5,000:1, 10,000:1, 25,000:1, 50,000:1, 100,000:1, 250,000:1, 500,000:1, and 750,000:1, or a range between any two of the preceding values, for example, between about 10:1 and about 750,000:1. For example, the ratio of concentrating volume 128 to culturing volume 130 may be at least about 10:1.

Each well 104 may be defined in culturing volume 130 by a culturing volume height 131, e.g., between neck 126 and bottom 110 along vertical well axis 122. Culturing volume height 131 may have a value in mm of one of, or one of about: 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2, or a range between any two of the preceding values, for example, from about 0.001 mm to about 2 mm or from about 0.1 mm to about 2 mm. For example, culturing volume height 131 in culturing volume 130 may be between about 0.1 mm and about 2 mm.

In some embodiments, neck 126 may meet bottom 110 in each well 104, wherein, e.g., total concentrating volume 128 may be equal to total volume 120, neck cross-sectional area 126′ may be equal to bottom surface area 110′, and culturing volume 130 and culturing volume height 131 may each have a value of zero.

In system 100, each well 104 may be characterized by a focusing factor and a notional focusing height, each of which may be related to concentrating volume 128 divided by neck cross-sectional area 126′. Because volume divided by area results in units of length, concentrating volume 128 divided by neck cross-sectional area 126′ may be contemplated in units of length characteristic of each well 104, e.g., the notional focusing height. The focusing height may be converted to the focusing factor, which is unitless, by dividing by unit length. For example, a focusing height of 25 mm divided by unit length of 1 mm corresponds to a unitless focusing factor of 25. Each well 104 may be characterized by the focusing factor at a value of about, at least about, or greater than one of: 25, 50, 75, 100, 125, 150, 175, 200, 250, 500, 750, 1,000, 1,500, 2,000, 2,500, 5,000, 7,500, 10,000, 15,000, 20,000, 30,000, 40,000, or 50,000, or a range between any two of the preceding values, for example, between about: 25-50,000, 50-50,000, 100-50,000, 200-50,000, 500-50,000, and the like. For example, each well 104 may be characterized by a focusing factor of at least about 50.

Each well 104 may be characterized by opening cross-sectional area 106′ being greater than neck cross-sectional area 126′. Each well 104 may be characterized by a ratio of opening cross-sectional area 106′ to neck cross-sectional area 126′ that is one of at least about 25:1, 49:1, 100:1, 144:1, 196:1, 256:1, 324:1, 400:1, 625:1, 900:1,1,600:1, 2,500:1, 3,600:1, 4,900:1, 6,400:1, 8,100:1, and 10,000:1, or a range between any two of the preceding values, for example, between about: 25:1 to 10,000:1, 100:1 to 10,000:1, and the like. For example, each well 104 may be characterized by a ratio of opening cross-sectional area 106′ to neck cross-sectional area 126′ of at least about 25:1.

Each well 104 may be characterized by an average horizontal dimension of opening 106 being greater than an average horizontal dimension of neck 126. Each well 104 may be characterized by a ratio of the average horizontal dimension of opening 106 divided by the average horizontal dimension of neck 126 being one of at least about 5:1, 7:1, 10:1, 12:1, 14:1, 16:1, 18:1, 20:1, 25:1, 30:1, 40:1, 50:1, 60:1, 70:1, 80:1, 90:1, and 100:1, or a range between any two of the preceding values, for example, between about: 5:1 to 100:1, 10:1 to 100:1, and the like. For example, each well 104 may be characterized by a ratio of the average horizontal dimension of opening 106 divided by the average horizontal dimension of neck 126 of at least about 5:1.

Each well 104 may be characterized by a ratio of neck cross-sectional area 126′ divided by bottom surface area 110′ that is one of at least about 1.2:1, 1.4:1, 1.7:1, 2:1, 2.25:1, 2.5:1, 3.24:1, 4:1, 9:1, 16:1, 25:1, 36:1, 49:1, 64:1, 81:1, and 100:1, e.g., at least about 1.2:1, or a range between any two of the preceding values, for example, between about 1.2:1 and about 100:1. For example, each well 104 may be characterized by a ratio of neck cross-sectional area 126′ divided by bottom surface area 110′ that is at least about 1.2:1. Each well 104 may be characterized by a ratio of neck cross-sectional area 126′ divided by bottom surface area 110′ that is, or is about, 1:1.

Each well 104 may be characterized by a ratio of bottom surface area 110′ divided by neck cross-sectional area 126′ that is one of at least about 1.2:1, 1.4:1, 1.7:1, 2:1, 2.25:1, 2.5:1, 3.24:1, 4:1, 9:1, 16:1, 25:1, 36:1, 49:1, 64:1, 81:1, and 100:1, e.g., at least about 1.2:1, or a range between any two of the preceding values, for example, between about 1.2:1 and about 100:1. For example, each well 104 may be characterized by a ratio of bottom surface area 110′ divided by neck cross-sectional area 126′ that is at least about 1.2:1. Each well 104 may be characterized by a ratio of bottom surface area 110′ divided by neck cross-sectional area 126′ that is, or is about, 1:1.

Each well 104 may be characterized by a ratio of an average horizontal dimension of neck 126 divided by the average horizontal dimension of bottom 110 that is one of at least about 1.1:1, 1.2:1, 1.3:1, 1.4:1, 1.5:1, 1.6:1, 1.8:1, 2:1, 3:1, 4:1, 5:1, 6:1, 7:1, 8:1, 9:1, and 10:1, e.g., at least about 1.1:1, or a range between any two of the preceding values, for example, between about 1.1:1 and about 10:1. For example, each well 104 may be characterized by the average horizontal dimension of neck 126 divided by the average horizontal dimension of bottom 110 that is at least about 1.1:1. Each well 104 may be characterized by a ratio of the average horizontal dimension of neck 126 divided by the average horizontal dimension of bottom 110 that is, or is about, 1:1.

Each well 104 may be characterized by a ratio of the average horizontal dimension of bottom 110 divided by the average horizontal dimension of neck 126 that is one of at least about 1.1:1, 1.2:1, 1.3:1, 1.4:1, 1.5:1, 1.6:1, 1.8:1, 2:1, 3:1, 4:1, 5:1, 6:1, 7:1, 8:1, 9:1, and 10:1, e.g., at least about 1.1:1, or a range between any two of the preceding values, for example, between about 1.1:1 and about 10:1. For example, each well 104 may be characterized by bottom 110 divided by the average horizontal dimension of neck 126 that is at least about 1.1:1. Each well 104 may be characterized by a ratio of bottom 110 divided by the average horizontal dimension of neck 126 that is, or is about, 1:1.

Each neck cross-sectional area 126′ may have a value in in μm² of one of about: 75, 100, 125, 150, 175, 200, 250, 500, 750, 1000, 1500, 2000, 2500, 5000, 7500, 10,000, 25,000, 50,000, 75,000, 100,000, 125,000, 150,000, 175,000, 200,000, 250,000, 500,000, 750,000, and 800,000, or a range between any two of the preceding values, for example, between about 75 μm² and about 800,000 μm², between about 7500 μm² and about 125,000 μm², and the like. Neck 126 may be characterized by an average horizontal dimension in μm of one of about 10, 20, 30, 40, 50, 75, 100, 125, 150, 175, 200, 250, 300, 400, 500, 600, 700, 800, 900, 1000, or a range between any two of the preceding values, for example, between about 10 μm and about 1000 μm or between about 100 μm and about 400 μm. Neck 126 may be characterized by a shape in horizontal cross-section that is one or more of: round, oval, polygonal, rounded polygonal, or irregular.

At least a portion of each well 104 may decrease in average horizontal dimension along wall 118 from opening 106 towards neck 126. At least a portion of each well 104 along wall 118 between opening 106 and neck 126 may define one of: a cylinder, a truncated cone, a truncated paraboloid, a hyperboloid, and a truncated hyperboloid. The truncated cone may be characterized by an apex truncated by neck 126. The truncated parabola may be truncated by neck 126, for example, between a focus of the paraboloid and a notional vertex of the paraboloid. The truncated hyperboloid may be truncated by neck 126 above, below, or at a vertex of the hyperboloid, a focus of the parabola and a notional vertex of the parabola. At least a portion of each well 104 along wall 118 between opening 106 and neck 126 may define a cylinder. Wall 118 in each well 104 between opening 106 and neck 126 may be characterized by a shape in horizontal cross-section that is one of: round, oval, polygonal, rounded polygonal, or irregular.

For example, FIG. 1A is a sketch in vertical cross-section showing that wall 118 may define a truncated cone that decreases in diameter from opening 106 towards neck 126 and is truncated by neck 126. FIG. 1C is a sketch in vertical cross-section showing that wall 118 may define a truncated paraboloid that decreases in diameter from opening 106 towards neck 126 and is truncated by neck 126. FIG. 1D is a sketch in vertical cross-section showing that wall 118 may define a truncated hyperboloid that decreases in diameter from opening 106 towards neck 126. FIG. 1E is a sketch in vertical cross-section showing that wall 118 may define a hyperboloid that extends from opening 106 to bottom 110, with neck 126 located at the vertex of the hyperboloid. FIG. 1F is a sketch in vertical cross-section showing that wall 118 may define two truncated sections, e.g., truncated cones, that each decrease in diameter from opening 106 towards neck 126. Each truncated cone described herein may have an independently selected angle with respect to the vertical well axis that has an absolute value of about one of: 5°, 10°, 20°, 25°, 30°, 40°, 45°, 50°, 60°, 65°, 70°, 80°, or 85°, or a range between any two of the preceding values, for example, 5° to 85°, 10° to 80°, 20° to 70°, 30° to 60°, 40° to 50°, or 45° to 55°.

At least a portion of each well 104 may decrease in average horizontal dimension along wall 118 from neck 126 to bottom 110. Well 104 may decrease in average horizontal dimension along wall 118 from neck 126 to bottom 110. At least a portion of each well 104 may define a cylinder or may decrease in average horizontal dimension along wall 118 from neck 126 to bottom 110 to define a portion of at least one of: a cone, a paraboloid, and a hyperboloid. Each well 104 may decrease in average horizontal dimension along wall 118 from neck 126 to bottom 110 to define a portion of at least one of: a cone, a paraboloid, and a hyperboloid. At least a portion of wall 118 between neck 126 and bottom 110 may define a cone characterized by an apex truncated at bottom 110. Wall 118 between neck 126 and bottom 110 may define a cone characterized by an apex truncated at bottom 110.

At least a portion of each well 104 may increase in average horizontal dimension along wall 118 from neck 126 to bottom 110. Well 104 may increase in average horizontal dimension along wall 118 from neck 126 to bottom 110. At least a portion of each well 104 may increase in average horizontal dimension along wall 118 from neck 126 to bottom 110 to define a portion of at least one of: a cone, a paraboloid, and a hyperboloid. Each well 104 may increase in average horizontal dimension along wall 118 from neck 126 to bottom 110 to define a portion of at least one of: a cone, a paraboloid, and a hyperboloid. At least a portion of wall 118 between neck 126 and bottom 110 may define a cone characterized by an apex truncated at neck 110. Wall 118 between neck 126 and bottom 110 may define a cone characterized by an apex truncated at neck 126.

One or more portions of each well 104 along wall 118 between neck 126 and bottom 110 may each independently define at least a portion of one of: a cylinder, a cone, a paraboloid, and a hyperboloid. For example, wall 118 may define a cylinder extending from neck 126 to bottom 110. Wall 118 may define a truncated cone extending upward from bottom 110 to an apex of the truncated cone; and a cylinder extending from the apex of the truncated cone upward to neck 126. Wall 118 may define a cone extending upward from an apex truncated by bottom 110; and a cylinder extending from neck 126 downward to meet the cone. Wall 118 may define a truncated cone extending downward from neck 126 to an apex of the truncated cone; and a cylinder extending from the apex of the truncated cone downward to bottom 110. Wall 118 may define a cone extending downward from an apex truncated by neck 126; and a cylinder extending from bottom 110 upward to meet the cone.

At least a portion of each well 104 may define a first cone along wall 118 between opening 106 and neck 126, an apex of the first cone truncated at neck 126. At least a portion of each well 104 may define a second cone between neck 126 and bottom 110. An apex of the second cone may be truncated by neck 126. An apex of the second cone may be truncated by bottom 110. The first cone may be characterized by a first angle with respect to vertical well axis 122, and the second cone may be characterized by a second angle with respect to vertical well axis 122. The first and second angles may be different. The first and second angles may be the same.

For example, FIG. 1A is a sketch in vertical cross-section showing that wall 118 may define a truncated cone that increases in diameter from bottom 110 towards neck 126. FIG. 1G is a sketch in vertical cross-section showing that wall 118 may define a truncated cone that decreases in diameter from bottom 110 towards neck 126. FIG. 1H is a sketch in vertical cross-section showing that wall 118 may define a truncated paraboloid that decreases in diameter from bottom 110 towards neck 126. FIG. 1I is a sketch in vertical cross-section showing that wall 118 may define a truncated hyperboloid that decreases in diameter from bottom 110 towards neck 126. Each truncated cone described herein may have an independently selected angle with respect to the vertical well axis that has an absolute value of about one of: 5°, 10°, 20°, 25°, 30°, 40°, 45°, 50°, 60°, 65°, 70°, 80°, or 85°, or a range between any two of the preceding values, for example, 5° to 85°, 10° to 80°, 20° to 70°, 30° to 60°, 40° to 50°, or 45° to 55°.

Wall 118 in each well 104 may independently be characterized in each of concentrating volume 128 and culturing volume 130 by a shape in horizontal cross-section that is one or more of: round, oval, polygonal, rounded polygonal, or irregular (not shown).

System 100 may include one or more wells 104 in each cell culture device 102 in a number that may be an integer of about, or at least about one of: 1, 2, 4, 6, 8, 12, 16, 24, 32, 48, 64, 96, 128, 256, 384, 1536, 3456, or 9600, for example, at least about 2, at least about 8, at least about 96, at least about 1536. Wells 104 may be incorporated into cell culture device 102 as a single row of wells, for example as a single row multi-well plate, a linear multi-well strip or tape, and the like (not shown). Wells 104 may be incorporated into cell culture device 102 as a regular or staggered array of rows and columns, for example, a 3:2 array such as 6×4=24 wells, a 1:1 array of 4×4=16 wells, and the like (not shown). System 100 may further include a frame 124 that holds at least one cell culture device 102. For example, frame 124 may hold 8 single row cell culture devices of 8 wells each to form a system of 8×8=64 wells, single cell culture devices in rows x columns of 8×8 to give a system of 64 wells, and the like (not shown). FIG. 1J is a perspective drawing showing cell culture device 102 as a multi-well plate with a regular array of 8×12=96 wells, held in frame 124.

Opening 106 of each well 104 may be characterized by an opening cross-sectional area 106′ that is greater than bottom surface area 110′. Opening cross-sectional area 106′ may be characterized in a ratio to bottom surface area 110′ of about, at least about, or greater than one of: 50:1, 60:1, 70:1, 80:1, 90:1, 100:1, 125:1, 150:1, 175:1, 200:1, 250:1, 300:1, 400:1, 500:1, 750:1, 1,000:1, 1,500:1, 2,000:1, 2,500:1, 5,000:1, 7,500:1, 10,000:1, 15,000:1, 20,000:1, or 25,000:1, or a range between any two of the preceding values, for example, between greater than 50:1 and about 25,000:1, between about 100:1 and about 25,000:1, between about 250:1 and about 25,000:1, between about 400:1 and about 25,000:1, and the like. For example, opening cross-sectional area 106′ may be characterized in a ratio to bottom surface area 110′ of about 50:1.

Opening cross-sectional area 106′ may have a surface area in mm² of one of about: 0.2, 0.25, 0.5, 0.75, 1, 1.5, 2, 2.5, 3, 5, 7.5, 10, 20, 30, 40, 50, 60, 70, 80, 90, 100, 125, 150, and 175, or a range between any two of the preceding values, for example, between about: 0.2-175 mm², 0.2-80 mm², 0.2-20 mm², 0.75-3 mm², 0.75-20 mm², 0.75-80 mm², and the like. For example, opening cross-sectional area 106′ may have a horizontal surface area in mm² of from about 0.75 to 325.

Opening cross-sectional area 106′ may have an average horizontal dimension in mm, e.g., a diameter, of one of about 0.5, 0.6, 0.7, 0.8, 0.9, 1, 1.5, 2. 2.5, 3, 4, 5, 6, 7, 7.5 8, 9, 10, 12.5, and 15, or a range between any two of the preceding values, for example, between about: 0.5-15 mm, 0.5-10 mm, 0.5-5 mm, 1-2 mm, 1-5 mm, 1-10 mm, and the like. For example, opening cross-sectional area 106′ may have an average horizontal dimension in mm of between about 0.5 to about 10. In some embodiments, each average horizontal dimension described herein may be a diameter.

Bottom 110 may be characterized by bottom surface area 110′ having a value in μm² of one of about: 75, 100, 125, 150, 175, 200, 250, 500, 750, 1000, 1500, 2000, 2500, 5000, 7500, 10,000, 25,000, 50,000, 75,000, 100,000, 125,000, 150,000, 175,000, 200,000, 250,000, 500,000, 750,000, and 800,000, or a range between any two of the preceding values, for example, between about 250 μm² and about 800,000 μm². For example, bottom surface area 110′ may be from about 75 μm² to about 800,000 μm².

Bottom 110 may be characterized by bottom surface area 110′ having an average horizontal dimension in μm of one of about 10, 20, 30, 40, 50, 75, 100, 125, 150, 175, 200, 250, 300, 400, 500, 600, 700, 800, 900, 1000, or a range between any two of the preceding values, for example, between about 10 μm and about 1000 μm. For example, bottom 110 may be characterized by bottom surface area 110′ having an average horizontal dimension in μm of between about 10 to about 1000.

Bottom surface area 110′ may be characterized by a surface profile that is one of: convex, concave, irregular and planar. For example, bottom surface area 110′ may be planar.

Bottom 110 at bottom surface area 110′ may be characterized by a roughness profile that meets an optically smooth criterion, expressed as D_{RMS}<λ/(8 cos θ), where D_{RMS} is the surface roughness (e.g., root-mean-square roughness distance measured from an average surface height of bottom 110 at bottom surface area 110′ in nanometers), λ is the wavelength of the light, and θ is the angle of incidence of the light. For example, bottom 110 at bottom surface area 110′ may be characterized at λ=400 nm and θ=45-90 degrees with respect to bottom 110, by a value of D_{RMS} in nanometers that is less than about one of: 400, 350, 300, 250, 200, 150, 100, 75, 50, 25, 20, 15, 10, 7.5, 5, 4, 3, 2, or 1, or a range between any two of the preceding values, for example, between about: 400-1 nm, 100-1 nm, 10-1 nm, and the like. For example, bottom 110 at bottom surface area 110′ may be characterized by a roughness profile having a D_{RMS} less than about 400 nm.

Bottom surface area 110′ may be characterized by a shape in horizontal cross section that is one or more of: round, oval, polygonal, rounded polygonal, or irregular (not shown).

Bottom 110 may be formed integral to cell culture device 102. Bottom 110 may be formed of a window material distinct from a material of cell culture device 102 and inserted into cell culture device 102 to meet wall 118 (not shown). Bottom 110 may be formed of the window material in contact with lower surface 112 of cell culture device 102 (not shown).

Each well 104 may include a window located to permit analysis of three-dimensional micro-tissue 116 received at bottom surface area 110′. The window may include a material characterized by at least partial transparency effective to permit one or more of imaging and spectroscopy. For example, bottom 110 may form the window.

At least a portion of bottom 110 may include a material characterized by at least partial transparency effective to permit one or more of: imaging and spectroscopy. For example, bottom 110 may include one or more of: polystyrene, polycarbonate, glass, quartz, and sapphire. Imaging and spectroscopy may include, e.g.: direct imaging; microscopy, e.g., confocal microscopy, or ultraviolet, visible, infrared, luminescence, or fluorescence microscopy; ultraviolet, visible, infrared, luminescence, or fluorescence spectroscopy, combinations thereof, and the like.

Each well 104 may include a bottom surface layer (not shown) on at least a portion of bottom 110 and bottom surface area 110′ facing well 104. The bottom surface layer may include one or more of: a biocompatible coating, a coating configured for mitigating cell adhesion, a coating configured for enhancing cell adhesion, and an antireflection coating.

Total volume 120 may be characterized by a value in μL of one of about, or at least one of about: 10, 15, 20, 25, 30, 40, 50, 60, 70, 80, 90, 100, 125, 150, 175, 200, 225, 250, 500, and 1,000, or a range between any two of the preceding values, for example, between about: 10-1,500 μL, 10-1,000 μL, 10-250 μL, 50-225 μL, 10-200 μL, 25-125 μL, 50-100 μL, and the like. For example, total volume 120 may be characterized by a volume in μL of between about 10 and about 1500.

Each well 104 may be characterized by a well height 105 between opening 106 and bottom surface area 110′ along vertical well axis 122. The height of each well 104 may be a value in mm of one of about, or at least one of about: 1, 2, 2.5, 3, 4, 5, 6, 7, 7.5, 8, 9, 10, 11, 12, 12.5, 13, 14, 15, 17.5, 20, 22.5, and 25, or a range between any two of the preceding values, for example, between about: 1-25 mm, 1-20 mm, 2-15 mm, 5-12.5 mm, 7.5-10 mm, and the like. For example, well height 105 of each well 104 may be a value in mm of between about 1 and about 25.

At least a portion of each well 104 along wall 118 from opening 106 towards bottom 110 may define one of: a cylinder, a truncated cone, a truncated paraboloid, a hyperboloid, and a truncated hyperboloid. At least a portion of each well 104 may decrease in average horizontal dimension along wall 118 from opening 106 towards bottom 110. For example, at least a portion of wall 118 from opening 106 towards bottom 110 may define a truncated cone that decreases in diameter towards bottom 110. For example, wall 118 may define a truncated cone that extends from opening 106 to an apex truncated at bottom 110. At least a portion of each well 104 along wall 118 from opening 106 towards bottom 110 may define a cylinder (not shown). At least a portion of each well 104 along wall 118 from opening 106 towards bottom 110 may define a truncated cone or ledge defined by a restricted horizontal dimension. The truncated cone or ledge may be effective to restrict insertion of an object defined by a diameter greater than the restricted horizontal dimension, for example, a pipette tip, a lid seal, and the like.

Each well 104 may be characterized by a shape in horizontal cross section that is one or more of: round, oval, polygonal, rounded polygonal, or irregular.

Cell culture device 102 may include one or more of: polystyrene, polycarbonate, polyethylene, polypropylene, polyoxymethylene, a cyclic polyolefin, a fluoropolymer, glass, quartz, sapphire, silicon, and a silicone polymer. At least a portion of cell culture device 102 may include a material that is opaque. For example, at least a portion of cell culture device 102 that forms wall 118 of each well 104 may be opaque.

Each well 104 may include a wall surface layer on at least a portion of wall 118. The wall surface layer may include one or more of: a biocompatible coating, a coating configured for mitigating cell adhesion, a covalently attached monolayer, a metal film, and an irradiated layer.

Features described herein for system 100 may be independently selected, e.g., from among the various corresponding features described herein. Features described herein for each well 104 may be independently selected. For example, features described herein for each well 104 may be different. Features described herein for each well 104 may be the same. System 100 may be operated using any aspect of the method for described herein.

In various embodiments, a system for obtaining a three-dimensional micro-tissue, e.g., for characterization, is provided. The system may include at least one cell culture device 102. Cell culture device 102 may include one or more wells 104. For example, cell culture device 102 may be a well plate or a microfluidic plate. Each well 104 may include opening 106 at upper surface 108 of cell culture device 102. Each well 104 may include bottom 110 located towards lower surface 112 of cell culture device 102. Bottom 110 may be characterized by bottom surface area 110′ inside well 104. Bottom surface area 110′ may be configured to receive aggregating cells, cell clusters, and/or tissue fragments 114 to form three-dimensional micro-tissue 116. Each well 104 may include a wall 118 extending from opening 106 to bottom surface area 110′ to define total volume 120. Wall 118 may extend from opening 106 to bottom surface area 110′ without defining a neck in well 104.

Each well 104 may be characterized by any aggregation factor value described herein, e.g., greater than about 100. For example, each well 104 may be characterized by the aggregation factor at a value of about, at least about, or greater than one of: 100, 125, 150, 175, 200, 250, 300, 400, 500, 600, 700, 750, 800, 900, 1,000, 1,500, 2,000, 2,500, 5,000, 7,500, 10,000, 15,000, 20,000, 30,000, 40,000, 50,000, 75,000, or 100,000, or a range between any two of the preceding values, for example, between greater than 100 and about 100,000, between about 200 and about 100,000, between about 500 and about 50,000, between about 800 and about 75,000, and the like.

Each well 104 may be described with reference to a vertical well axis 122. Vertical well axis 122 may extend from a centroid of opening 106 in a direction down through a centroid of bottom 110. Wall 118 may extend in rotational symmetry about vertical well axis 122. One or more of upper surface 108, lower surface 112, bottom 110, and bottom surface area 110′ may be horizontal with respect to vertical well axis 122.

The system may include one or more wells 104 in each cell culture device 102 in a number that may be an integer of about, or at least about one of: 1, 2, 4, 6, 8, 12, 16, 24, 32, 48, 64, 96, 128, 256, 384, 1536, 3456, or 9600, for example, at least about 2, at least about 8, at least about 96, at least about 1536. Wells 104 may be incorporated into cell culture device 102 as a single row of wells, for example as a single row multi-well plate, a linear multi-well strip or tape, and the like (not shown). Wells 104 may be incorporated into cell culture device 102 as a regular or staggered array of rows and columns, for example, a 3:2 array such as 6×4=24 wells, a 1:1 array of 4×4=16 wells, and the like (not shown).

The system may further include a frame 124 that holds at least one cell culture device 102. For example, frame 124 may hold 8 single row cell culture devices of 8 wells each to form a system of 8×8=64 wells, single cell culture devices in rows x columns of 8×8 to give a system of 64 wells, and the like (not shown). Features described herein for each cell culture device 102 may be independently selected. For example, features described herein for each cell culture device 102 may be different. Features described herein for each cell culture device 102 may be the same.

Opening 106 of each well 104 may be characterized by an opening cross-sectional area 106′ that is greater than bottom surface area 110′. Opening cross-sectional area 106′ may be characterized in a ratio to bottom surface area 110′ of about, at least about, or greater than one of: 50:1, 60:1, 70:1, 80:1, 90:1, 100:1, 125:1, 150:1, 175:1, 200:1, 250:1, 300:1, 400:1, 500:1, 750:1, 1,000:1, 1,500:1, 2,000:1, 2,500:1, 5,000:1, 7,500:1, 10,000:1, 15,000:1, 20,000:1, or 25,000:1, or a range between any two of the preceding values, for example, between greater than 50:1 and about 25,000:1, between about 100:1 and about 25,000:1, between about 250:1 and about 25,000:1, between about 400:1 and about 25,000:1, and the like. For example, opening cross-sectional area 106′ may be characterized in a ratio to bottom surface area 110′ of about 50:1.

Opening cross-sectional area 106′ may have a surface area in mm² of one of about: 0.2, 0.25, 0.5, 0.75, 1, 1.5, 2, 2.5, 3, 5, 7.5, 10, 20, 30, 40, 50, 60, 70, 80, 90, 100, 125, 150, and 175, or a range between any two of the preceding values, for example, between about: 0.2-175 mm², 0.2-80 mm², 0.2-20 mm², 0.75-3 mm², 0.75-20 mm², 0.75-80 mm², and the like. For example, opening cross-sectional area 106′ may have a horizontal surface area in mm² of from about 0.75 to 325.

Opening cross-sectional area 106′ may have an average horizontal dimension in mm, e.g., a diameter, of one of about 0.5, 0.6, 0.7, 0.8, 0.9, 1, 1.5, 2. 2.5, 3, 4, 5, 6, 7, 7.5 8, 9, 10, 12.5, and 15, or a range between any two of the preceding values, for example, between about: 0.5-15 mm, 0.5-10 mm, 0.5-5 mm, 1-2 mm, 1-5 mm, 1-10 mm, and the like. For example, opening cross-sectional area 106′ may have an average horizontal dimension in mm of between about 0.5 to about 10. In some embodiments, each average horizontal dimension described herein may be a diameter.

Bottom 110 may be characterized by bottom surface area 110′ having a value in μm² of one of about: 75, 100, 125, 150, 175, 200, 250, 500, 750, 1000, 1500, 2000, 2500, 5000, 7500, 10,000, 25,000, 50,000, 75,000, 100,000, 125,000, 150,000, 175,000, 200,000, 250,000, 500,000, 750,000, and 800,000, or a range between any two of the preceding values, for example, between about 250 μm² and about 800,000 μm². For example, bottom surface area 110′ may be from about 75 μm² to about 800,000 μm².

Bottom 110 may be characterized by bottom surface area 110′ having an average horizontal dimension in μm of one of about 10, 20, 30, 40, 50, 75, 100, 125, 150, 175, 200, 250, 300, 400, 500, 600, 700, 800, 900, 1000, or a range between any two of the preceding values, for example, between about 10 μm and about 1000 μm. For example, bottom 110 may be characterized by bottom surface area 110′ having an average horizontal dimension in μm of between about 10 to about 1000.

Bottom surface area 110′ may be characterized by a surface profile that is one of: convex, concave, irregular and planar. For example, bottom surface area 110′ may be planar.

Bottom 110 at bottom surface area 110′ may be characterized by a roughness profile subject to an optically smooth criterion, expressed as D_{RMS}<λ/(8 cos θ), where D_{RMS} is the surface roughness (e.g., root-mean-square roughness distance measured from an average surface height of bottom 110 at bottom surface area 110′ in nanometers), λ is the wavelength of the light, and θ is the angle of incidence of the light. For example, bottom 110 at bottom surface area 110′ may be characterized at λ=400 nm and θ=45-90 degrees with respect to bottom 110, by a value of D_{RMS} in nanometers that is less than about one of: 400, 350, 300, 250, 200, 150, 100, 75, 50, 25, 20, 15, 10, 7.5, 5, 4, 3, 2, or 1, or a range between any two of the preceding values, for example, between about: 400-1 nm, 100-1 nm, 10-1 nm, and the like. For example, bottom 110 at bottom surface area 110′ may be characterized by a roughness profile having a standard deviation less than about 400 nm.

Bottom surface area 110′ may be characterized by a shape in horizontal cross-section that is one or more of: round, oval, polygonal, rounded polygonal, or irregular (not shown).

Bottom 110 may be formed integral to cell culture device 102. Bottom 110 may be formed of a window material distinct from a material of cell culture device 102 and inserted into cell culture device 102 to meet wall 118 (not shown). Bottom 110 may be formed of the window material in contact with lower surface 112 of cell culture device 102 (not shown).

Each well 104 may include a window located to permit analysis of three-dimensional micro-tissue 116 received at bottom surface area 110′. The window may include a material characterized by at least partial transparency effective to permit one or more of imaging and spectroscopy. For example, bottom 110 may form the window.

At least a portion of bottom 110 may include a material characterized by at least partial transparency effective to permit one or more of: imaging and spectroscopy. Imaging and spectroscopy may include, e.g.: direct imaging; microscopy, e.g., confocal microscopy, or ultraviolet, visible, infrared, luminescence, or fluorescence microscopy; ultraviolet, visible, infrared, luminescence, or fluorescence spectroscopy, combinations thereof, and the like. For example, bottom 110 may include one or more of: polystyrene, polycarbonate, glass, quartz, and sapphire.

Each well 104 may include a bottom surface layer (not shown) on at least a portion of bottom 110 and bottom surface area 110′ facing well 104. The bottom surface layer may include one or more of: a biocompatible coating, a coating configured for mitigating cell adhesion, a coating configured for enhancing cell adhesion, and an antireflection coating.

Total volume 120 may be characterized by a value μL it of one of about, or at least one of about: 10, 15, 20, 25, 30, 40, 50, 60, 70, 80, 90, 100, 125, 150, 175, 200, 225, 250, 500, and 1,000, or a range between any two of the preceding values, for example, between about: 10-1,500 μL, 10-1,000 μL, 10-250 μL, 50-225 μL, 10-200 μL, 25-125 μL, 50-100 μL, and the like. For example, total volume 120 may be characterized by a volume μL it of between about 10 and about 1500.

Each well 104 may be characterized a height between opening 106 and bottom surface area 110′ along vertical well axis 122. The height of each well 104 may be a value in mm of one of about, or at least one of about: 1, 2, 2.5, 3, 4, 5, 6, 7, 7.5, 8, 9, 10, 11, 12, 12.5, 13, 14, 15, 17.5, 20, 22.5, and 25, or a range between any two of the preceding values, for example, between about: 1-25 mm, 1-20 mm, 2-15 mm, 5-12.5 mm, 7.5-10 mm, and the like. For example, the height of each well 104 may be a value in mm of between about 1 and about 25.

At least a portion of each well 104 along wall 118 from opening 106 towards bottom 110 may define one of: a cylinder, a truncated cone, a truncated paraboloid, a hyperboloid, and a truncated hyperboloid. At least a portion of each well 104 may decrease in average horizontal dimension along wall 118 from opening 106 towards bottom 110. For example, at least a portion of wall 118 from opening 106 towards bottom 110 may define a truncated cone that decreases in diameter towards bottom 110. For example, wall 118 may define a truncated cone that extends from opening 106 to an apex truncated at the bottom 110. At least a portion of each well 104 along wall 118 from opening 106 towards bottom 110 may define a cylinder (not shown). At least a portion of each well 104 along wall 118 from opening 106 towards bottom 110 may define a truncated cone or ledge defined by a restricted horizontal dimension. The truncated cone or ledge may be effective to restrict insertion of an object defined by a diameter greater than the restricted horizontal dimension, for example, a pipette tip, a lid seal, and the like.

Each well 104 may be characterized by a shape in horizontal cross-section that is one or more of: round, oval, polygonal, rounded polygonal, or irregular.

Cell culture device 102 may include one or more of: polystyrene, polycarbonate, polyethylene, polypropylene, polyoxymethylene, a cyclic polyolefin, a fluoropolymer, glass, quartz, sapphire, silicon, and a silicone polymer. At least a portion of cell culture device 102 may include a material that is opaque. For example, at least the portion of cell culture device 102 that forms wall 118 of each well 104 may be opaque.

Each well 104 may include a wall surface layer on at least a portion of wall 118. The wall surface layer may include one or more of: a biocompatible coating, a coating configured for mitigating cell adhesion, a covalently attached monolayer, a metal film, and an irradiated layer.

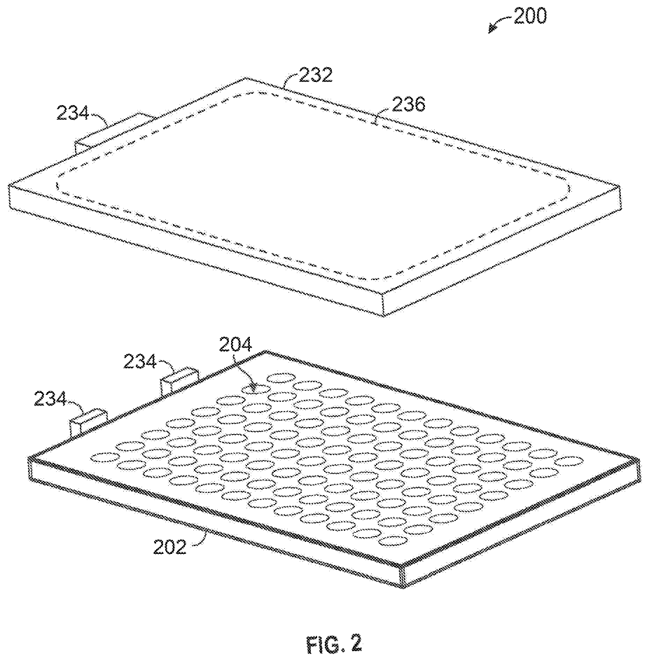

FIG. 2 is a perspective view of an example system 200. System 200 includes a cell culture device 202 with one or more, e.g., a plurality of wells 204. System 200 may include a lid 232 configured to cover cell culture device 202.

Lid 232 may include any suitable material or combination of materials, such as: glass; quartz; sapphire; silicon; a polymer such as polycarbonate, polyethylene, polypropylene, polyoxymethylene, a cyclic polyolefin, a fluoropolymer, a silicone polymer, polyvinylidene chloride, polyethylene terephthalate; a textile that is one or more of synthetic, natural, impregnated, nonwoven; and the like.

Lid 232 may include a material characterized by at least partial transparency effective to permit one or more of imaging and spectroscopy. Lid 232 may be opaque. Lid 232 may filter or block light, e.g., in desired wavelengths such as ultraviolet, visible, or infrared. Lid 232 may be rigid; semi rigid; or flexible, e.g., as a plastic film.

System 200 may further include at least one fastener 234 configured to at least temporarily fasten lid 232 to cell culture device 202. For example, fastener 234 may include a hinge, a friction fit, a clip, an elastic band, a latch, and the like. System 200 may include with two or more fasteners 234, such as a hinge and latch combination. For example, in the hinge and latch combination, the hinge may temporarily or permanently couple lid 232 to cell culture device 202. For example, in the hinge and latch combination, the latch may be configured to permit lid 232 to be reversibly latched closed, or unlatched to swing open from cell culture device 202.

System 200 may further include at least one sealing element 236. Sealing element 236 may be configured to at least partly seal between lid 232 and cell culture device 202. Sealing element 236 may be configured to at least partly seal between lid 232 and each well 204. Sealing element 236 may be configured to at least partly seal between lid 232 and independently a plurality of wells 204. Suitable commercial seals and sealing materials, e.g., silicone rubber, are widely available that provide for reversible or permanent sealing and compatibility with cell culture. In some embodiments, sealing element 236 may be integral with lid 232, for example, configured together as an elastomeric silicone polymer lid. In some embodiments, sealing element 236 may be integral with cell culture device 202, for example, with sealing element 236 as an elastomeric silicone polymer over-mold on cell culture device 202.

Lid 232 may be configured to hermetically seal each cell culture device 202. Lid 232 may be configured to hermetically seal each well 204. Lid 232 may be configured to seal each cell culture device 202 with a modulated flow seal. Lid 232 may be configured to hermetically seal each well 204 with a modulated flow seal.

The modulated flow seal may be effective to provide a modulated gas exchange equivalent to a circular opening characterized by a diameter in μm of less than about one of: 1000, 750, 500, 250, 100, 90, 80, 70, 60, 50, 40, 30, 20, 10, 5, 4, 3, 2, or 1, or a range between any two of the preceding values, for example, modulated about: 1-1000 μm, 1-250 μm, 1-100 μm, 20-100 μm, and the like. The modulated gas exchange may be provided by a single opening in the lid at each cell culture device or each well, e.g., by a circular opening. The modulated gas exchange may be provided by one or more openings of circular or other shapes, e.g., a plurality of irregular perforations. The restricted gas flow may be provided by a permeable, semipermeable, or selectively permeable material included by lid 232, such as a porous expanded polytetrafluoroethylene membrane, a permeable gel, and the like. Suitable commercial permeable, semipermeable, selectively permeable materials are widely available that provide for controlled permeation of air, oxygen, water, water vapor, and other species. For example, the modulated flow seal may be effective to provide a modulated gas exchange equivalent to that of a circular opening less than about 1000 μm in diameter. Further, for example, lid 232 may include material configured to modulate permeability to one or more of: air, oxygen (O₂), and water vapor. Lid 232 may include material configured to modulate permeability to one or more of: air, oxygen (O₂), water, water vapor, viruses, bacteria, and particulates.

Features described herein for system 200 may be independently selected, e.g., from among the various corresponding features described for system 100. Features described herein for each well 204 may be independently selected, e.g., from the features described for each well 104. For example, features described herein for each well 204 may be different. Features described herein for each well 204 may be the same. System 200 may be operated using any aspect of the method for described herein for obtaining a three-dimensional micro-tissue, e.g., for characterization.

In various embodiments, a system is provided for obtaining a three-dimensional micro-tissue, e.g., for characterization, is provided. The system may include at least one cell culture device. The cell culture device may include a plurality of wells. Each well may include an opening at an upper surface of the cell culture device characterized by an opening cross-sectional area. Each well may include a bottom located towards a lower surface of the cell culture device. The bottom may be characterized by bottom surface area inside each well. The bottom may be characterized by transparency effective to permit imaging or spectroscopy of each well, e.g., from below the lower surface of the cell culture device. Each well may include a neck located between the opening and the bottom. Each well may include a wall extending from the opening to the bottom. The wall may define a total volume between the opening and the bottom. The wall may define a concentrating volume between the neck and the opening. The wall may define a culturing volume between the neck and the bottom. Each well may be characterized by a total volume divided by the bottom surface area divided by unit length to define any aggregation factor described herein, for example, a value greater than 400. Each well may be characterized by any volume ratio of the concentrating volume divided by the culturing volume described herein, for example, at least about 10:1. Each well may be characterized by the cross-sectional area of the opening divided by the bottom surface area to define any concentration ratio described herein, for example, a concentration ratio greater than 50:1. Each well may be characterized by any ratio of the opening cross-sectional area to the neck cross-sectional area described herein, for example, a ratio of at least about 25:1.

In various embodiments, a system for obtaining a three-dimensional micro-tissue is provided. The system may include a cell culture device. The cell culture device may include a plurality of wells. Each well may include an opening at an upper surface of the device. The opening may be characterized by an opening cross-sectional area. Each well may include a bottom located towards a lower surface of the device. The bottom may be characterized by a bottom surface area inside the well. The bottom surface area may include a planar portion. The bottom may be characterized by transparency effective to permit imaging or spectroscopy inside each well, e.g., from below the lower surface of the cell culture device. Each well may include a wall extending from the opening to the bottom. The wall may define a total volume between the opening and the bottom. The wall may define a neck located below the opening. The well may define a neck characterized by a neck cross-sectional area parallel to the opening. The wall may define a concentrating volume between the neck and the opening. The wall may define a culturing volume between the neck and the bottom. Each well may be characterized by an aggregation factor of greater than 800. The aggregation factor may correspond to the total volume divided by the bottom surface area divided by a unit length.

Each well may be characterized by one or more of: the aggregation factor between 800 and 40,000; a ratio of the concentrating volume divided by the culturing volume of at least about 10:1; a ratio of the opening cross-sectional area divided by the bottom surface area greater than 50:1; a ratio of the opening cross-sectional area to the neck cross-sectional area of at least about 25:1; and the concentrating volume divided by a neck cross-sectional area divided by unit length to define a focusing factor, the focusing factor being at least about 50.

The neck in each well may be characterized by one or more of: the neck cross-sectional area in μm² from about 75 to 750,000; and an average horizontal dimension in μm of between about 10 to about 1000.

The concentrating volume in each well may be between about 10 μL and about 1500 μL, and the culturing volume in each well may be between about 1 μL and about 250 μL.

In some embodiments, at least a portion of the wall in each well between the opening and the neck may define at least one of: a cylinder, a cone with a truncated apex at the neck, a paraboloid with a truncated vertex at the neck, and a hyperboloid that decreases in diameter towards the neck. The wall in each well between the opening and the neck may define two or more such shapes, for example, the wall may define a cylinder extending down from the opening, and a cone truncated at the neck, the cone increasing in diameter and extending upwards to the cylinder. Each well along the wall between the neck and the bottom may define one of: a cylinder, a truncated cone, a truncated paraboloid, a hyperboloid, and a truncated hyperboloid.

In several embodiments, the opening of each well may be characterized by one or more of: a horizontal surface area in mm² of from about 0.75 to 325; and an average horizontal dimension in mm of between about 0.5 to about 10. The bottom in each well may be characterized by one or more of: the bottom surface area in μm² of from about 7500 to 125,000; and an average horizontal dimension in μm of between about 100 to about 400. The neck in each well may be characterized by one or more of: the neck cross-sectional area in μm² of from about 7500 to 125,000; and an average horizontal dimension in μm of between about 100 to about 400.

In various embodiments, the cell culture may include one or more of: polystyrene, polycarbonate, polyethylene, polypropylene, polyoxymethylene, a cyclic polyolefin, a fluoropolymer, glass, quartz, sapphire, silicon, and a silicone polymer. Each well may include one or more of: a biocompatible coating, a coating configured for mitigating cell adhesion, a covalently attached monolayer, a metal film, and an irradiated layer. At least a portion of the cell culture device may include a material that is opaque.

The system may further include a lid configured to cover each cell culture device. The system may include a sealing element configured to provide a hermetic or modulated flow seal for one or more of: each cell culture device between the lid and the cell culture device; each well between the lid and the cell culture device; and each well independently between the lid and the cell culture device.

In some embodiments, at least one well may be loaded with one or more of: a suspension of cells, cell clusters, and/or tissue fragments in the concentrating volume; the three-dimensional micro-tissue on the bottom in the culturing volume; and a concentration gradient in the three-dimensional micro-tissue, the concentration gradient corresponding to one or more of: a gas, a metabolite, a nutrient, a biomolecule, an imaging contrast agent, and a therapy for evaluation. The cells, cell clusters, tissue fragments, and three-dimensional micro-tissue may include one of human cells; primary tumor cells; or cells from a tumor line.

Features described herein for each system may be independently selected, e.g., from among the various corresponding features described for systems such as 100 and 200. Features described herein for each well may be independently selected, e.g., from the features described for each well such as 104 and 204. For example, features described herein for each well may be different. Features described herein for each well may be the same. The system may be operated using any aspect of the method for described herein for obtaining a three-dimensional micro-tissue, for characterizing a three-dimensional micro-tissue, and the like.

In various embodiments, the systems described herein may include a suspension of cells, cell clusters, and/or tissue fragments 114 in each described volume. The systems described herein may include three-dimensional micro-tissue 116 on bottom 110, e.g., in culturing volume 130. The systems described herein may include a concentration gradient in three-dimensional micro-tissue 116. The concentration gradient may correspond to one or more of: a gas, a metabolite, a nutrient, a biomolecule, an imaging contrast agent, and a therapy for evaluation. Cells, cell clusters, and/or tissue fragments 114 may include one or more of: human cells; primary tumor cells; and cells from a tumor line. The system may include the three-dimensional micro-tissue including human primary tumor cells. The system may include the concentration gradient in the three-dimensional micro-tissue.

In various embodiments, a method is provided for obtaining a three-dimensional micro-tissue, e.g., for characterization. The method may include providing a total volume that includes a suspension of cells, cell clusters, and/or tissue fragments. The method may include aggregating the cells, cell clusters, and/or tissue fragments from the total volume of the suspension to a bottom surface area. The aggregating may be conducted according to an aggregation factor greater than 100. The aggregating may be effective to obtain the three-dimensional micro-tissue.

The method may be conducted independent of each system described herein. The method may include using any aspect of any system described herein.

In the method, the aggregation factor and a corresponding aggregation height may be related to the total volume divided by the bottom surface area. Dividing volume by area results in units of length, or the aggregation height. The aggregation height may be converted to the aggregation factor, which is unitless, by dividing by unit length. For example, an aggregation factor of 100 mm divided by unit length of 1 mm corresponds to a unitless aggregation factor of 100. The aggregating may be conducted using the aggregation factor at a value of about, at least about, or greater than one of: 100, 125, 150, 175, 200, 250, 500, 750, 1,000, 1,500, 2,000, 2,500, 5,000, 7,500, 10,000, 15,000, 20,000, 30,000, 40,000, or 50,000, or a range between any two of the preceding values, for example, greater than 100, between greater than 100 and about 50,000, between about 200 and about 50,000, between about 500 and about 50,000, between about 750 and about 50,000, and the like. For example, in the method, the aggregation factor may be from greater than 100 to about 50,000.

The method may include aggregating the cells, cell clusters, and/or tissue fragments to the bottom surface area. The bottom surface area may have a value in μm² of one of about: 75, 100, 125, 150, 175, 200, 250, 500, 750, 1000, 1500, 2000, 2500, 5000, 7500, 10,000, 25,000, 50,000, 75,000, 100,000, 125,000, 150,000, 175,000, 200,000, 250,000, 500,000, 750,000, and 800,000, or a range between any two of the preceding values, for example, between about 250 μm² and about 800,000 μm². For example, bottom surface area 110′ may be from about 75 μm² to about 800,000 μm². The bottom surface area may be characterized as having an average horizontal dimension in μm of one of about 10, 20, 30, 40, 50, 75, 100, 125, 150, 175, 200, 250, 300, 400, 500, 600, 700, 800, 900, 1000, or a range between any two of the preceding values, for example, between about 10 μm and about 1000 μm.

The method may include aggregating the cells to the bottom surface area characterized by a surface profile that is one of: convex, concave, irregular and planar. The method may include aggregating the cells to the bottom surface area characterized by a surface profile that is planar. The method may include aggregating the cells to the bottom surface area characterized by optical smoothness in the wavelength range used for imaging or spectroscopy, for example, a roughness profile having a D_{RMS} as described herein in nanometers that is less than about one of: 400, 350, 300, 250, 200, 150, 100, 75, 50, 25, 20, 15, 10, 7.5, 5, 4, 3, 2, or 1, or a range between any two of the preceding values, for example, between about: 400-1 nm, 100-1 nm, 10-1 nm, and the like. For example, the bottom surface area may be characterized by D_{RMS} having a standard deviation less than about 400 nm.

The method may further include characterizing the three-dimensional micro-tissue by one or more of imaging and spectroscopy. The method may include using the bottom surface area including a surface of a window material characterized by at least partial transparency. The method may further include characterizing the three-dimensional micro-tissue through the window material by one or more of imaging and spectroscopy. Imaging and spectroscopy may include, e.g.: direct imaging; microscopy, e.g., confocal microscopy, or ultraviolet, visible, infrared, luminescence, or fluorescence microscopy; ultraviolet, visible, infrared, luminescence, or fluorescence spectroscopy, combinations thereof, and the like. The method may include using the window material including one or more of: polystyrene, polycarbonate, glass, quartz, and sapphire.

The method may include providing the total volume that may be characterized by a value μL it of one of about, or at least one of about: 10, 15, 20, 25, 30, 40, 50, 60, 70, 80, 90, 100, 125, 150, 175, 200, 225, 250, 500, and 1,000, or a range between any two of the preceding values, for example, between about: 10-1,500 μL, 10-1,000 μL, 10-250 μL, 50-225 μL, 10-200 μL, 25-125 μL, 50-100 μL, and the like. For example, the total volume may be characterized by a volume in μL of between about 10 and about 1500.

The method may include aggregating over a distance of one of about, or at least one of about: 1, 2, 2.5, 3, 4, 5, 6, 7, 7.5, 8, 9, 10, 11, 12, 12.5, 13, 14, 15, 17.5, 20, 22.5, and 25, or a range between any two of the preceding values, for example, between about: 1-25 mm, 1-20 mm, 2-15 mm, 5-12.5 mm, 7.5-10 mm, and the like. For example, the method may include aggregating over a distance in mm of between about 1 and about 25.

The aggregating may include concentrically aggregating with respect to a vertical well axis. The method may include aggregating under gravity. The method may include aggregating using centrifugation.

The method may include aggregating the cells from a concentrating volume of the suspension through a neck cross-sectional area to a culturing volume above the bottom surface area. Aggregating through the neck cross-sectional area may be conducted according to a focusing factor. In the method, the focusing factor and a focusing height may be related to concentrating volume divided by a neck cross-sectional area. Dividing volume by area results in units of length, or the focusing height. The focusing height may be converted to the focusing factor, which is unitless, by dividing by unit length. For example, a focusing height of 25 mm divided by unit length of 1 mm corresponds to a unitless focusing factor of 25. In the method, the aggregation may be characterized by the focusing factor at a value of about, at least about, or greater than one of: 25, 50, 75, 100, 125, 150, 175, 200, 250, 500, 750, 1,000, 1,500, 2,000, 2,500, 5,000, 7,500, 10,000, 15,000, 20,000, 30,000, 40,000, or 50,000, or a range between any two of the preceding values, for example, between about: 25-50,000, 50-50,000, 100-50,000, 200-50,000, 500-50,000, and the like. For example, the aggregation may be characterized by the focusing factor of at least about 50.

The aggregating may be conducted using a ratio of the neck cross-sectional area to the bottom surface area that is one of at least about 1.2:1, 1.4:1, 1.7:1, 2:1, 2.25:1, 2.56:1, 3.24:1, 4:1, 9:1, 16:1, 25:1, 36:1, 49:1, 64:1, 81:1, and 100:1, e.g., at least about 1.2:1, or a range between any two of the preceding values, for example, between about 1.2:1 and about 100:1. For example, the ratio of neck cross-sectional area divided by the bottom surface area may be at least about 1.2:1. The ratio of neck cross-sectional area divided by the bottom surface area may be, or be about, 1:1.

The aggregating may be conducted using a ratio of the bottom surface area to the neck cross-sectional area that is one of at least about 1.2:1, 1.4:1, 1.7:1, 2:1, 2.25:1, 2.56:1, 3.24:1, 4:1, 9:1, 16:1, 25:1, 36:1, 49:1, 64:1, 81:1, and 100:1, e.g., at least about 1.2:1, or a range between any two of the preceding values, for example, between about 1.2:1 and about 100:1. For example, the ratio of bottom surface area divided by the neck cross-sectional area may be at least about 1.2:1. The ratio of the bottom surface area divided by the beck cross-sectional area may be, or be about, 1:1.

The aggregating may be conducted using the neck cross-sectional area at a value in in μm² of one of about: 75, 100, 125, 150, 175, 200, 250, 500, 750, 1000, 1500, 2000, 2500, 5000, 7500, 10,000, 25,000, 50,000, 75,000, 100,000, 125,000, 150,000, 175,000, 200,000, 250,000, 500,000, 750,000, and 800,000, or a range between any two of the preceding values, for example, between about 75 μm² and about 800,000 μm².

The method may include aggregating the cells from the concentrating volume at a value μL it of one of about, or at least one of about: 10, 15, 20, 25, 30, 40, 50, 60, 70, 80, 90, 100, 125, 150, 175, 200, 225, 250, 500, 750, 1,000, 1,250, and 1,500, or a range between any two of the preceding values, for example, between about: 10-1,500 μL, 10-1,000 μL, 10-250 μL, 50-225 μL, 10-200 μL, 25-125 μL, 50-100 μL, and the like.

The method may include aggregating the cells from the concentrating volume of the suspension through the neck cross-sectional area over a distance along a vertical well axis of at least about: 0.5, 1, 1.5, 2, 2.5, 3, 4, 5, 6, 7, 7.5, 8, 9, 10, 11, 12, 12.5, 13, 14, 15, 17.5, 20, 22.5, and 25, or a range between any two of the preceding values, for example, between about: 0.5-25 mm, 0.5-20 mm, 1-15 mm, 2-12 mm, 2-10 mm, 2-4 mm, and the like.

The method may include using the culturing volume a value μL it of one of about, or at least one of about: 1, 2, 2.5, 3, 4, 5, 6, 7, 7.5, 8, 9, 10, 15, 20, 25, 30, 40, 50, 60, 70, 80, 90, 100, 125, 150, 175, 200, 225, and 250, or a range between any two of the preceding values, for example, between about: 1-250 μL, 1-200 μL, 1-150 μL, 1-50 μL, 1-25 μL, and the like. For example, the culturing volume may be between about 1 μL and about 250 μL.

The method may include aggregating the cells from the neck cross-sectional area to the culturing volume above the bottom surface area over a distance along a vertical well axis in mm of one of about, or at least one of about: 0.1, 0.125, 0.15, 0.175, 0.2, 0.25, 0.3, 0.4, 0.5, 0.6, 0.7, 0.8, 0.9, 1, 1.1, 1.2, 1.25, 1.5, 1.75, and 2, or a range between any two of the preceding values, for example, between about: 0.1-2 mm, 0.2-2 mm, 0.2-1 mm, 0.5-1 mm, 1-2 mm, and the like. For example, the distance may be between about 0.1 mm and about 2 mm.

The aggregating may include using a ratio of the concentrating volume divided by the culturing volume that is one of at least about 10:1, 15:1, 20:1, 25:1, 50:1, 75:1, 100:1, 250:1, 500:1, 750:1, 1,000:1, 1,500:1, 2,500:1, 5,000:1, 10,000:1, 25,000:1, 50,000:1, 100,000:1, 250,000:1, 500,000:1, and 750,000:1, or a range between any two of the preceding values, for example, between about 10:1 and about 750,000:1. For example, the aggregating may include using a ratio of the concentrating volume divided by the culturing volume of at least about 10:1.

The method may include covering one or more of the total volume, the concentrating volume, the culturing volume, and the three-dimensional micro-tissue. The covering may include hermetic sealing. The covering may include modulating gas exchange to a flow equivalent to that of a circular opening less than about 1000 μm in diameter. The covering may include modulating gas exchange of one or more of: air, oxygen (O₂), and water vapor. The covering may include modulating exposure to one or more of: air, oxygen (O₂), water, water vapor, viruses, bacteria, and particulates.

The method may include providing, forming, or allowing the formation of a concentration gradient in the three-dimensional micro-tissue. The concentration gradient may correspond to one or more of: a gas, a metabolite, a nutrient, a biomolecule, and a therapy for evaluation. The concentration gradient may increase in a direction upwards from the bottom surface area. Providing, forming, or allowing the formation concentration gradient to the three-dimensional micro-tissue may include contacting the three-dimensional micro-tissue with one or more of: the gas, the metabolite, the nutrient, the biomolecule, and the therapy for evaluation; and allowing the concentration gradient to be established in the three-dimensional micro-tissue.

The therapy for evaluation may include one or more of: an anticancer agent; a therapeutic agent used as an adjunct in cancer therapy; and a compound suspected of modulating cancer therapy.

Suitable anticancer agents for testing may include any of the hundreds of anticancer agents and combinations thereof known to the art for treating cancer. Anticancer agents may include alkylating agents, for example: nitrogen mustards, such as mechlorethamine, chlormethine, cyclophosphamide, melphalan, chlorambucil, ifosfamide and busulfan; nitrosoureas such as N-nitroso-N-methylurea (MNU), carmustine (BCNU), lomustine (CCNU), semustine (MeCCNU), fotemustine, bendamustine, estramustine, and streptozotocin; tetrazines such as dacarbazine, mitozolomide and temozolomide; aziridines such as thiotepa, mytomycin and diaziquone (AZQ); platins such as cisplatin, carboplatin, nedaplatin, and oxaliplatin; and non-classical alkylating agents such as dacarbazine, procarbazine, and hexamethylmelamine; derivatives thereof; and the like. Anticancer agents may include antimetabolites, for example: anti-folates such as methotrexate and pemetrexed; fluoropyrimidines such as fluorouracil and capecitabine; deoxynucleoside analogues such as cytarabine, gemcitabine, decitabine, azacitidine, fludarabine, nelarabine, cladribine, clofarabine, and pentostatin; thiopurines such as thioguanine and mercaptopurine; derivatives thereof; and the like. Anticancer agents may include anti-microtubule agents, for example: vinca alkaloids such as vincristine, vinblastine, vinorelbine, vindesine, and vinflunine; taxanes such as cabazitaxel, paclitaxel, and docetaxel; lignans and derivatives such as podophyllotoxin, etoposide, and teniposide; derivatives thereof; and the like. Anticancer agents may include topoisomerase inhibitors, for example: topoisomerase I inhibitors such as irinotecan and topotecan; topoisomerase II poisons such as etoposide, doxorubicin, mitoxantrone and teniposide; topoisomerase II inhibitors such as novobiocin, merbarone, and aclarubicin; derivatives thereof; and the like. Anticancer agents may include cytotoxic antibiotics, for example: anthracyclines such as doxorubicin, daunorubicin, epirubicin, idarubicin, pirarubicin, aclarubicin, and mitoxantrone; bleomycin; mitomycin; dactinomycin; mitoxantrone; actinomycin; derivatives thereof; and the like. Anticancer agents may include nucleoside analogs; for example: azacitidine; capecitabine; carmofur; cladribine; clofarabine; cytarabine; decitabine; floxuridine; fludarabine; fluorouracil; gemcitabine; mercaptopurine; nelarabine; pentostatin; tegafur; tioguanine; derivatives thereof; and the like. Anticancer agents may include, for example: antifolates such as methotrexate; pemetrexed; raltitrexed; hydroxycarbamide; derivatives thereof; and the like. Anticancer agents may include other agents, for example: anagrelide; arsenic trioxide; asparaginase; denileukin diftitox; vemurafenib; derivatives thereof; and the like.

Anticancer agents may include, for example, targeted antineoplastics. Anticancer agents may include tyrosine kinase inhibitors, for example: afatinib; aflibercept; axitinib; bosutinib; crizotinib; dasatinib; erlotinib; gefitinib; imatinib; lapatinib; nilotinib; pazopanib; ponatinib; regorafenib; ruxolitinib; sorafenib; sunitinib; vandetanib; derivatives thereof; and the like. Anticancer agents may include mTOR inhibitors, for example: everolimus; temsirolimus; tacrolimus; derivatives thereof; and the like. Anticancer agents may include retinoids, for example: alitretinoin; bexarotene; isotretinoin; tamibarotene; tretinoin; derivatives thereof; and the like. Anticancer agents may include immunomodulatory agents, for example: lenalidomide; pomalidomide; thalidomide; derivatives thereof; and the like. Anticancer agents may include histone deacetylase inhibitors, for example: panobinostat; romidepsin; valproate; vorinostat; derivatives thereof; and the like. Anticancer agents may include species that may have reduced or no activity due to the nature of the three-dimensional microtissue, but such species may be analyzed in the presence of the three-dimensional microtissue nevertheless. For example, the three-dimensional microtissue may exist independent of an immune system, and anticancer agents may include monoclonal antibodies, e.g.: alemtuzumab; bevacizumab; cetuximab; denosumab; gemtuzumab ozogamicin; ibritumomab tiuxetan; ipilimumab; nivolumab; ofatumumab; panitumumab; pembrolizumab; pertuzumab; rituximab; tositumomab; trastuzumab; derivatives thereof; and the like. Anticancer agents may include any other agent known to the art, such as oncolytic viruses. Anticancer agents may be agents expected or suspected of having anticancer activity, for example, new agents under discovery and development for anticancer activity.

Various therapeutic agents are known to the art for use as an adjunct in cancer therapy, for example, to assist anticancer activity, to mitigate anticancer agent side effects, to mitigate other effects of the cancer, and the like. Therapeutic agents for use as an adjunct in cancer therapy may include, for example: anti-inflammatory agents; analgesic agents; antiemetic agents; hormone therapeutics, e.g., testosterone, estrogen, estradiol, blocking agents thereof, and the like; selective androgen receptor modulators; selective estrogen receptor modulators; cannabinoids; osteoporosis therapeutics; diabetes therapeutics; and the like.

In some embodiments, it may be desirable to test a compound suspected of modulating cancer therapy. The compound suspected of modulating cancer therapy may include any existing or new compound being screened for anticancer activity. The compound suspected of modulating cancer therapy may include, for example, a compound that is present or expected to be present in a subject in addition to cancer therapy. The compound that is present or expected to be present in a subject in addition to cancer therapy may be, for example, a pharmaceutical administered to the subject for another condition; a non-prescribed drug or dietary supplement that may be present in the subject; a compound that is a product of the subject's metabolism or of a disease process in the subject; an environmental compound, such as a compound in the subject's food or the subject's environment; and the like.

The method may include treating the three-dimensional micro-tissue with an adjuvant therapy. The adjuvant therapy may include one or more of: supraphysiological temperature, subphysiological temperature, sonotherapy, electrochemotherapy, radiation; and the like.

The suspension of cells, cell clusters, and tissue/tissue fragments may be any such material derivable from multicellular organisms, such as eukaryotic organisms, e.g., mammals, and particularly humans. Such materials may be derived from healthy and/or diseased tissue, e.g., cancerous tissue, particularly solid tumors. As used herein tumor material means any material derived from tumors, e.g., solid tumors. Tumor material may be derived from tumor cells; tumor cell clusters; or tumor tissue or fragments thereof. The tumor material may be fresh, frozen or defrosted. The tumor material may include benign tumor material as well as malignant tumor material.

The tumor material may be derived from a naturally occurring system, e.g., a tumor material containing a body fluid or components derived from a body fluid, e.g., isolated body fluid components or processed body fluids. Tumor material may be derived from a biological fluid such as serum, saliva, urine, bile, lymphatic fluid, cerebrospinal fluid and/or other body fluids.

Tumor material may be derived from a naturally occurring system containing a tissue or components derived from a tissue, e.g., isolated tissue components or processed tissue components. A tissue or a component derived therefrom may be of various origins, for example muscle tissue, gastrointestinal tissue, heart tissue, liver tissue, kidney tissue, epithelial tissue, connective tissue, neuronal tissue, and the like.

The tumor material may be derived from any cancer, e.g., cancers that form solid tumors. For example, tumor material may be derived from tissue of mesenchymal origin such as lymphomas or leukemias, from tissue of epithelial origin, such as lung cancer, pancreatic cancer, colorectal cancer, ovarian cancer, and the like. The tumor material is may include one of floating tumor cells and tumor cells isolated from tumor tissue.

The tumor material may be derived from human or non-human subjects. The tumor material may be derived from human or non-human mammals. The tumor material may be derived from a human subject. The suspension may be any suspension containing tumor material described herein. Methods for obtaining a suspension containing tumor material are known to the art, including suitable parameters, such as buffer system, temperature and pH for the suspension to be used with the present invention. The suspension may include a number of cells of about one of: 100, 250, 500, 750, 1000, 5,000, 10,000, 25,000, 50,000, 75,000, or 100, 000, or a range between any two of the preceding values, for example, between about 100-100,000 cells, 1,000-100,000 cells, 10,000-100,000 cells, and the like.

The incubation of the sedimented cells may be conducted using any methods known to the art for cultivating eukaryotic cells. It is within the knowledge of a person skilled in the art to choose the optimal parameters, such as buffer system, temperature and pH for the incubation of the tumor material sediment in accordance with the present invention. The incubation may be conducted at a temperature in ° C. of one of about: 5, 10, 15, 20, 25, 30, 35, 37, 40, or 45, or a range between any two of the preceding values, for example, 5-40° C., 15-37° C., 27-37° C., and the like. For example, the cells may be incubated at 37° C.

The incubation may be carried out for a time in hours of at least one of: 2, 4, 6, 12, 18, 24, 36, 48, 72, 96, 120, 144, or 16, or a range between any two of the preceding values, for example, from about: 6-144 hours, 6-120 hours, 6-96 hours, 6-72 hours, 6-48 hours, 6-36 hours, 6-24 hours, 6-12 hours, 24-144 hours, 24-120 hours, 24-72 hours, 24-48 hours, or 24-36 hours.

In various embodiment, a method for characterizing a three-dimensional micro-tissue is provided. The method may include providing a system for obtaining a three-dimensional micro-tissue. The system may include a cell culture device. The cell culture device may include a plurality of wells. Each well may include an opening at an upper surface of the device. The opening may be characterized by an opening cross-sectional area. Each well may include a bottom located towards a lower surface of the device. The bottom may be characterized by a bottom surface area inside the well. The bottom surface area may include a planar portion. The bottom may be characterized by transparency effective to permit imaging or spectroscopy inside each well, e.g., from below the lower surface of the cell culture device. Each well may include a wall extending from the opening to the bottom. The wall may define a total volume between the opening and the bottom. The wall may define a neck located below the opening. The well may define a neck characterized by a neck cross-sectional area parallel to the opening. The wall may define a concentrating volume between the neck and the opening. The wall may define a culturing volume between the neck and the bottom. Each well may be characterized by an aggregation factor of greater than 800. The aggregation factor may correspond to the total volume divided by the bottom surface area divided by a unit length. The method may include providing each well with a suspension of cells, cell clusters, and/or tissue fragments. The method may include aggregating the cells, cell clusters, and/or tissue fragments from the suspension to a bottom surface area according to the aggregation factor. The aggregating may be effective to obtain the three-dimensional micro-tissue. The method may include characterizing the three-dimensional micro-tissue inside each well from below the lower surface of the cell culture device.

Characterizing the three-dimensional micro-tissue may include one or more of: direct imaging and spectroscopy. Imaging and spectroscopy may include, e.g.: direct imaging; microscopy, e.g., confocal microscopy, or ultraviolet, visible, infrared, luminescence, or fluorescence microscopy; ultraviolet, visible, infrared, luminescence, or fluorescence spectroscopy, combinations thereof, and the like. The aggregating may include using one or both of gravity and centrifugation. The cells, cell clusters, and/or tissue fragments may include one of human cells; primary tumor cells; or cells from a tumor line.

The method may further include covering each well. The covering may include one of: hermetic sealing; and modulating exposure of each well to one or more of: air, oxygen (O₂), water, water vapor, viruses, bacteria, and particulates.

Characterizing the three-dimensional micro-tissue may further include contacting the three-dimensional micro-tissue in each well with a therapy for evaluation that is independently selected for each well. The therapy for evaluation may include one or more of: an anticancer agent; a therapeutic agent used as an adjunct in cancer therapy; and a compound suspected of modulating cancer therapy. Characterizing the three-dimensional micro-tissue inside each well may include characterizing the therapy for evaluation according to therapeutic parameters independently selected for each well. “Independently selected” for each well means that therapeutic parameters of the therapy for evaluation may be varied between wells by any aspect desired to be evaluated for the therapy, such that the three-dimensional micro-tissue may be characterized by response to the independently selected therapeutic parameters. For example, different wells may be contacted with the therapy for evaluation using different dosages or different dosage schedules. Further, the therapy for evaluation may be varied in composition between wells, e.g., to test different anticancer agents, or, e.g., to test different relative amounts of the anticancer agents, therapeutic agents, and compounds desired for evaluation. Likewise, therapeutic parameters of the adjuvant therapy may be independently selected for each well.

The method may include providing a concentration gradient in the three-dimensional micro-tissue. The concentration gradient may correspond to one or more of: a gas, a metabolite, a nutrient, a biomolecule, a therapy for evaluation an adjuvant therapy, and the like.

The method may include treating the three-dimensional micro-tissue with an adjuvant therapy comprising one or more of: supraphysiological temperature, subphysiological temperature, sonotherapy, electrochemotherapy, and radiation.

In various embodiments, a method for obtaining a three-dimensional micro-tissue is provided. The method may include providing a system for obtaining a three-dimensional micro-tissue. The system may include a cell culture device. The cell culture device may include a plurality of wells. Each well may include an opening at an upper surface of the device. The opening may be characterized by an opening cross-sectional area. Each well may include a bottom located towards a lower surface of the device. The bottom may be characterized by a bottom surface area inside the well. The bottom surface area may include a planar portion. The bottom may be characterized by transparency effective to permit imaging or spectroscopy inside each well, e.g., from below the lower surface of the cell culture device. Each well may include a wall extending from the opening to the bottom. The wall may define a total volume between the opening and the bottom. The wall may define a neck located below the opening. The well may define a neck characterized by a neck cross-sectional area parallel to the opening. The wall may define a concentrating volume between the neck and the opening. The wall may define a culturing volume between the neck and the bottom. Each well may be characterized by an aggregation factor of greater than 800. The aggregation factor may correspond to the total volume divided by the bottom surface area divided by a unit length. The method may include providing each well with a suspension of cells, cell clusters, and/or tissue fragments. The method may include aggregating the cells, cell clusters, and/or tissue fragments from the suspension to a bottom surface area according to the aggregation factor. The aggregating may be effective to obtain the three-dimensional micro-tissue.

The present invention may form a three-dimensional micro-tissue. The parameters of the wells disclosed herein, e.g., wells 104 and 204, may provide concentrating and aggregating cells effective to form the three-dimensional micro-tissue. Without wishing to be bound by theory, the three-dimensional micro-tissue may recapitulate, for example, one or more of: three-dimensional cell-cell interactions, culture gradients, from the top to the bottom, of species found under physiological conditions such as a gas, a metabolite, a nutrient, a biomolecule, and a therapy for evaluation. Accordingly, the three-dimensional micro-tissue of the invention may provide an effective model for tissue behavior under such concentration gradients, for example, to simulate the concentration gradients inside a tumor.

Further, by providing the concentration gradient from the top to the bottom, with an analysis window at bottom 110, the lowest concentration of the gradient may be directly monitored for effects on cell behavior. Three-dimensional effects may not be obtained in 2D tissue models. Conventional 3D tissue sphereoids may be challenging to image at the low concentration core, especially for high resolution imaging. For example, in tumor models it may be desirable to image the core for the effect of lower concentrations of therapy for evaluation and/or oxygen. By contrast, in the present invention, a hypoxic, low-drug portion of the three-dimensional micro-tissue may be against the transparent bottom of the well, which may substantially facilitate imaging, especially by contrast with the difficulty of imaging the core of conventional spheroid models.

The invention may be configured to be compatible with conventional cell culture device readers, robotic liquid handlers, and other cell culture device analysis apparatuses effective to provide convenient assessment of cellular responses. In particular, the multi-well format may provide high throughput drug screening of physiologically-relevant micro-tissues.

Each system and cell culture device described herein may be configured with standard ANSI/SBS dimensions for compatibility with conventional cell culture device readers, robotic liquid handlers, and other cell culture device analysis devices. The compatible multi-well format may provide high throughput drug screening of physiologically-relevant micro-tissues.

The disclosure of the present application includes several embodiments, which may share common properties and features. The properties and features of one embodiment may be combined with properties and features of other embodiments. Similarly, a single property or feature or combination of properties or features in any embodiment may constitute a further embodiment.

Recitation of ranges of values herein are intended to illustrate each separate value falling within the range, and unless otherwise indicated herein, each individual value is incorporated into the specification as if it were individually recited herein.

The term “about” refers to a range of values of plus or minus 20% of a specified value. For example, the phrase “about 200” includes plus or minus 20% of 200, or from 160 to 240, unless specifically indicated otherwise or contradicted by context.

Ranges may be expressed herein as from “about” or “approximately” one particular value to “about” or “approximately” another particular value. When such a range is expressed, another embodiment includes between each such pair of particular values. Similarly, when values are expressed as approximations, by use of the antecedent “about” or “approximate” it will be understood that each particular value forms another embodiment.

It is understood that the endpoints of each of the ranges are significant both in relation to the other endpoint and independently of the other endpoint. It is also understood that there are a number of values disclosed, wherein each value is also disclosed as “about” that particular value in addition to the value itself. For example, if the value “10” is disclosed, then “about 10” is also disclosed. It is also understood that when a value is disclosed that is “less than or equal to the value” or “greater than or equal to the value” possible ranges between these values are also disclosed, as appropriately understood by the expert with ordinary skills in the art. For example, if the value “10” is disclosed the “less than or equal to 10” as well as “greater than or equal to 10” is also disclosed.

As used herein, recitation of lists of elements such as “at least one of A and B” or “one or more of A and B” using the conjunction “and” support conjunctive and disjunctive collections of the listed elements. For example, “at least one of A and B” includes: an embodiment having one of A; an embodiment having one of B; an embodiment having one of A and one of B; an embodiment having multiple instances of A, where the context permits; an embodiment having multiple instances of B, where the context permits; an embodiment having multiple instances of A where the context permits and one of B; an embodiment having multiple instances of B where the context permits and one of A; and an embodiment having multiple instances of A and multiple instances of B, where the context permits.

All methods described herein can be performed in any suitable order unless otherwise indicated herein or otherwise clearly contradicted by context. Units, prefixes, and symbols are denoted in their System International de Unites (SI) accepted form unless otherwise specified.

The following terms, as used herein, have the meanings ascribed to them unless specified otherwise.

As used herein, the term “administering” means the introduction of a composition into a container, well, cell, or tissue culture medium, or (as appropriate) onto cells, tissues or surfaces.

Such methods are well-known in the art. Any and all methods of introducing the composition are contemplated and the invention is not dependent on any particular means of introduction.

As used herein, the terms “agent” and “compound” are used interchangeably and mean any chemical compound, for example, a macromolecule or a small molecule disclosed herein. The agent may be naturally occurring (e.g. a herb or a natural product), non-naturally occurring, synthetic, purified, recombinant, and the like. An agent may be used alone or in combination with other agents in the methods described herein.

As used herein, a “micro-tissue” means a small aggregation of biological cells, typically of less than a million cells, including cells obtained from primary tissue or cell lines.

As used herein, “primary tissue” means tissue that was directly removed from an organism, for example by fine needle biopsy, core needle biopsy, or surgical biopsy, typically without further modification.

As used herein, “live primary tissue” means primary tissue in which some cells are alive. As used herein, “cell suspension” and “tissue suspension” may include cells, cell aggregates, tissue fragments, or other biological material suspended in an aqueous solution.

1. A system for obtaining a three-dimensional micro-tissue, comprising:
a cell culture device, comprising:
a plurality of wells, each well comprising:
an opening at an upper surface of the device, the opening characterized by an opening cross-sectional area;
a bottom located towards a lower surface of the device, the bottom characterized by a bottom surface area inside the well, the bottom surface area comprising a planar portion, and the bottom characterized by transparency effective to permit imaging or spectroscopy inside each well; and
a wall extending from the opening to the bottom, the wall defining:
a total volume between the opening and the bottom;
a neck located below the opening, the neck characterized by a neck cross-sectional area parallel to the opening;
a concentrating volume between the neck and the opening; and
a culturing volume between the neck and the bottom,
each well characterized by an aggregation factor of greater than 800, the aggregation factor corresponding to the total volume divided by the bottom surface area divided by a unit length.

2. The system of claim 1, each well characterized by one or more of:
the aggregation factor between 800 and 40,000;
a ratio of the concentrating volume divided by the culturing volume of at least about 10:1;
a ratio of the opening cross-sectional area divided by the bottom surface area greater than 50:1;
a ratio of the opening cross-sectional area to the neck cross-sectional area of at least about 25:1; and
the concentrating volume divided by a neck cross-sectional area divided by unit length to define a focusing factor, the focusing factor being at least about 50.

3. The system of claim 1, the concentrating volume in each well being between about 10 μL and about 1500 μL, and the culturing volume in each well being between about 1 μL and about 250 μL.

4. The system of claim 1,
at least a portion of the wall in each well between the opening and the neck defining at least one of: a cylinder, a cone with a truncated apex at the neck, a paraboloid with a truncated vertex at the neck, and a hyperboloid that decreases in diameter towards the neck; and
each well along the wall between the neck and the bottom defining one of: a cylinder, a truncated cone, a truncated paraboloid, a hyperboloid, and a truncated hyperboloid.

5. The system of claim 1, at least one of:
the opening of each well characterized by one or more of:
a horizontal surface area in mm² of from about 0.75 to 325; and
an average horizontal dimension in mm of between about 0.5 to about 10;
the bottom in each well characterized by one or more of:
the bottom surface area in μm² of from about 7500 to 125,000; and
an average horizontal dimension in μm of between about 100 to about 400; and
the neck in each well characterized by one or more of:
the neck cross-sectional area in μm² of from about 7500 to 125,000; and
an average horizontal dimension in μm of between about 100 to about 400.

6. The system of claim 1, the cell culture device comprising one or more of: polystyrene, polycarbonate, polyethylene, polypropylene, polyoxymethylene, a cyclic polyolefin, a fluoropolymer, glass, quartz, sapphire, silicon, and a silicone polymer.

7. The system of claim 1, at least a portion of the cell culture device comprising a material that is opaque.

8. The system of claim 1, each well comprising one or more of: a biocompatible coating, a coating configured for mitigating cell adhesion, a covalently attached monolayer, a metal film, and an irradiated layer.

9. The system of claim 1, further comprising:
a lid configured to cover each cell culture device;
a sealing element configured to provide a hermetic or modulated flow seal for one or more of:
each cell culture device between the lid and the cell culture device;
each well between the lid and the cell culture device; and
each well independently between the lid and the cell culture device.

10. The system of claim 1, at least one well being loaded with one or more of:
a suspension of cells, cell clusters, and/or tissue fragments in the concentrating volume;
the three-dimensional micro-tissue on the bottom in the culturing volume; and
a concentration gradient in the three-dimensional micro-tissue, the concentration gradient corresponding to one or more of: a gas, a metabolite, a nutrient, a biomolecule, an imaging contrast agent, and a therapy for evaluation.

11. A method for characterizing a three-dimensional micro-tissue, comprising:
providing a system for obtaining a three-dimensional micro-tissue, comprising:
a cell culture device, comprising:
a plurality of wells, each well comprising:
an opening at an upper surface of the device, the opening characterized by an opening cross-sectional area;
a bottom located towards a lower surface of the device, the bottom characterized by a bottom surface area inside the well, the bottom surface area comprising a planar portion, and the bottom characterized by transparency effective to permit imaging or spectroscopy inside each well from below the lower surface of the cell culture device; and
a wall extending from the opening to the bottom, the wall defining:
a total volume between the opening and the bottom;
a neck located below the opening, the neck characterized by a neck cross-sectional area parallel to the opening;
a concentrating volume between the neck and the opening; and
a culturing volume between the neck and the bottom,
each well characterized by an aggregation factor of greater than 800, the aggregation factor corresponding to the total volume divided by the bottom surface area divided by a unit length;
providing each well with a suspension of cells, cell clusters, and/or tissue fragments;
aggregating the cells, cell clusters, and/or tissue fragments from the suspension to the bottom surface area according to the aggregation factor, the aggregating being effective to obtain the three-dimensional micro-tissue; and
characterizing the three-dimensional micro-tissue inside each well from below the lower surface of the cell culture device.

12. The method of claim 11, characterizing the three-dimensional micro-tissue comprising one or more of: direct imaging; optical microscopy; confocal microscopy;
microscopy using ultraviolet, visible, infrared, luminescence, or fluorescence; ultraviolet spectroscopy; visible spectroscopy; infrared spectroscopy luminescence spectroscopy;
and fluorescence spectroscopy.

13. The method of claim 11, the aggregating comprising using one or both of gravity and centrifugation.

14. The method of claim 11, the cells, cell clusters, and/or tissue fragments comprising one of human cells; primary tumor cells; or cells from a tumor line.

15. The method of claim 11, further comprising covering each well, the covering comprising one of:
hermetic sealing;
modulating exposure of each well to one or more of: air, oxygen (O₂), water, water vapor, viruses, bacteria, and particulates.

16. The method of claim 11, further comprising contacting the three-dimensional micro-tissue in each well with a therapy for evaluation that is independently selected for each well, the therapy for evaluation comprising one or more of: an anticancer agent; a therapeutic agent used as an adjunct in cancer therapy; and a compound suspected of modulating cancer therapy.

17. The method of claim 16, characterizing the three-dimensional micro-tissue inside each well comprising characterizing the therapy for evaluation according to therapeutic parameters independently selected for each well.

18. The method of claim 11, comprising providing a concentration gradient in the three-dimensional micro-tissue, the concentration gradient corresponding to one or more of: a gas, a metabolite, a nutrient, a biomolecule, and a therapy for evaluation.

19. The method of claim 11, further comprising treating the three-dimensional micro-tissue with an adjuvant therapy comprising one or more of: supraphysiological temperature, subphysiological temperature, sonotherapy, electrochemotherapy, and radiation.

20. A method for obtaining a three-dimensional micro-tissue, comprising:
providing a system for obtaining a three-dimensional micro-tissue, comprising:
a cell culture device, comprising:
a plurality of wells, each well comprising:
an opening at an upper surface of the device, the opening characterized by an opening cross-sectional area;
a bottom located towards a lower surface of the device, the bottom characterized by a bottom surface area inside the well, the bottom surface area comprising a planar portion, and the bottom characterized by transparency effective to permit imaging or spectroscopy inside each well; and
a wall extending from the opening to the bottom, the wall defining:
a total volume between the opening and the bottom;
a neck located below the opening, the neck characterized by a neck cross-sectional area parallel to the opening;
a concentrating volume between the neck and the opening; and
a culturing volume between the neck and the bottom,
each well characterized by an aggregation factor of greater than 800, the aggregation factor corresponding to the total volume divided by the bottom surface area divided by a unit length;
providing each well with a suspension of cells, cell clusters, and/or tissue fragments; and
aggregating the cells, cell clusters, and/or tissue fragments from the suspension to a bottom surface area according to the aggregation factor,
the aggregating being effective to obtain the three-dimensional micro-tissue.
